(19) United States
(12) Patent Application Publication
(10) Pub. No.: US 2002/0119492 A1
(43) Pub. Date: Aug. 29, 2002

(54) Protein design automation for designing protein libraries with altered immunogenicity

(76) Inventors: Arthur J. Chirino, Camarillo, CA (US); Bassil I. Dahiyat, Altadena, CA (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/903,378

(22) Filed: Jul. 10, 2001

Related U.S. Application Data
(60) Provisional application No. 60/217,661, filed on Jul. 10, 2000.

(51) Int. Cl. G01N 33/53; G06F 19/00; G01N 33/48; G01N 33/50

(52) U.S. Cl. 435/7.1; 702/19

(57) ABSTRACT

The present invention relates to the use of a variety of computational methods for modulating the immunogenicity of proteins by identifying and then altering potential amino acid sequences that elicit an immune response in a host organism. In particular, proteins will be screened for MHC binding sequences, T cell epitopes and B cell epitopes.

This application claims the benefit of the priority date of U.S. Ser. No. 60/217,661, filed Jul. 10, 2000.

### FIELD OF THE INVENTION

The present invention relates to the use of a variety of computational methods for modulating the immunogenicity of proteins by identifying and then altering potential amino acid sequences that elicit an immune response in a host organism. In particular, proteins will be screened for MHC, T cell receptor, and B cell receptor binding sequences.

### BACKGROUND OF THE INVENTION

The distinction between what is foreign and what is “self” is of central importance during immune surveillance. The identification of proteins from foreign pathogens such as viruses and bacteria is a crucial step in adaptive immunity. Similar recognition processes occur during transplant organ rejection, in autoimmune disease and also can occur during the repeated or sustained systemic use of any exogenous protein or other macromolecule in humans. Adaptive immunity has two major arms: humoral immunity and cellular immunity. Immunoglobulin is the crux of the humoral immune response. As a cell surface receptor on B lymphocytes, immunoglobulin is responsible for instigating cellular responses as diverse as activation, differentiation, and programmed cell death. As secreted in antibody, immunoglobulin can bind a foreign antigen, neutralizing it directly or initiating steps necessary to arm and recruit effector systems such as complement or antibody dependent cell cytolysis by monocytic phagocytes (Fundamental Immunology, fourth edition, W. E. Paul, ed., Lippincott-Raven Publishers, 1999, Chapter 3, pp 37-74).

T cells are responsible for cellular immunity. T cells are known to directly kill target cells, to provide help for such killers, to activate other immune system cells (i.e., macrophages), to help B cells make an antibody response, to down modulate the activities of various immune system cells, and to secrete cytokines, chemokines, and other mediators. These activities are often mediated by distinct types of T cells, such as α:β T cells, type 1 and type 2 helper cells. Activation of a T cell occurs when it recognizes a particular antigen via receptors displayed on its surface (i.e. T cell receptors or TCRs). α:β T cells (i.e., CD8+ and CD4+ T cells) recognize an antigen only in association with one of the molecules encoded within the major histocompatibility complex (MHC) and then only if it is the appropriate allelic variant. This phenomenon is called MHC restriction (Fundamental Immunology, fourth edition, W. E. Paul, ed., Lippincott-Raven Publishers, 1999, Chapter 11, pp 367-409).

Major Histocompatibility Complex (MHC) molecules play a central role in the recognition process by binding polypeptide fragments derived from foreign proteins (antigens) and then presenting these peptides to receptors on the surface of T cells resulting in an immune response. The MHC molecule accomplishes its major role in immune recognition by satisfying two distinct molecular functions: the binding of peptide and the interaction with T cells, usually via the α:β T-cell receptor (TCR). The binding of peptides by an MHC I or MHC II molecule is the selective event that permits the cell expressing the MHC molecule (the antigen presenting cell, APC) to sample either its own proteins ( MHC I) or the proteins ingested from the immediate extracellular environment (MHC II) (Fundamental Immunology, fourth edition, W. E. Paul, ed., Lippincott-Raven Publishers, 1999, Chapter 8, pp 263-285).

The interaction between TCRs on one cell and complementary peptide-MHC complexes on another triggers a cascade of intercellular signals that depends on the identity of both the T cell and the antigen presenting cell. Ultimately, TCR-peptide-MHC recognition regulates immune responses including graft and tumor rejection, anti-viral cytolysis, and the recruitment and control of other immune cells such as antibody producing B cells (Madden, D. R., (1995) Annu. Rev. Immunol., 13:587-622)

MHC molecules are highly polymorphic and display allelic variation among different human populations (Buus, supra). Hundreds of MHC class I and II alleles are known, each exhibiting different binding affinities for specific antigenic peptide sequences. The structural basis for this allelic dependent peptide preference has been localized to differences in amino acid residues within the MHC peptide binding pocket (Buus, supra). X-ray crystal structure of MHC class I and II molecules bound to specific antigenic peptides reveal that peptide residues at the N and C termini, i.e., the anchor positions, are in close physical contact with the MHC class I binding pocket, while peptides bound to class II are more extended with additional peptide residues making contact with the MHC class II pocket (Buus, supra).

Extensive sequence analyses of peptides eluted from MHC molecules reveal some allele-specific amino acid preferences (Buus, supra). Databases consisting of thousands of peptide sequences know to bind MHC molecules have been compiled (Rammensee, H., et a. (1999) Immunogenetics, 50:213-219) and several techniques have been developed to analyze the sequences of full length proteins to predict the presence of potentially antigenic sequences (Hiemstra, H. S. et al. (2000) Curr. Op. Immunol., 12:80-84; Malios, R. R., (1999) Bioinformatics, 15:432-439; Sturniolo, T., et al. (1999) Nature Biotechnology, 17:555-561; Brusic, V., et al., (1998) Bioinformatics, 14:121-130; Mallios, R. R., (1998) J. Comp. Biol., 5:703-711; Savoie, C. J. et al. (1999) Pac Symp Biocomput, 182-9; Altuvia, Y., et al. (1997) Human Immunology, 58:1-11; Shastri, N. (1996) Curr. Op. Immunol., 8:271-277; Hammer, J. (1995) Curr. Op. Immunol., 7:263-269; Meister, G. E., et al. (1995) Vaccine, 13:581-591; Udaka, K., et al. (1995) J. Exp. Med., 181:20972108; Hammer, J. et al. (1994) Behring. Inst. Mitt. 94:124-132; Hammer, J., et al. (1994) J. Exp. Med., 180: 2353-2358; and, Rudenshky, A. Y., et al. (1991) Nature, 353:622-627). Although overall peptide binding affinity is sequence- and MHC-allele specific, the contribution of each peptide residue is independent of the identity of adjacent residues and can be summed individually (Altuvia, et al., supra). The presence of anchor residues and length of the MHC class I bound peptides has lead to better predictive models for MHC class I molecules than for MHC class II molecules (Abrams and Schlom, (2000) Curr. Op. Immunol., 12:85-91).

Although it is less clear which residues of an antigenic peptide are bound by the TCR, side-chain substitution experiments have mapped out the rough outlines of the TCR binding site on a number or peptide-MHC complexes. Typically, different TCRs are found to contact different, but overlapping, subsets of MHC and peptide side chains. TCR “footprints” are centered on the bound peptide and include MHC side chains on the tops of both α-helices that form the peptide-binding groove. Bound peptides clearly contribute prominently to TCR recognition despite the fact that a significant percentage of the peptide surface is buried. More recent results suggest that each amino acid in the peptide sequence contributes independently to the affinity of the MHC-peptide-TCR complex (Hemmer, B., et al., (1998), J. Immunol., 160.3631-3636).

An important component of humoral immunity are the diverse repertoires of antibodies (i.e., immunoglobulins) produced by B lymphocytes. Antigen contact with a specific B cell trigger the transmembrane signaling function of the B cell antigen receptor (BCR). This, in turn, induces early events in B cell activation, including increased expression of MHC class II molecules and formation of antibody secreting cells.

Reduction of polypeptide immunogenicity has been accomplished by using rational site directed mutagenesis (Meyer, et al., (2001) Protein Science 10:491-503), exhaustive site directed mutagenesis (Laroche, et al., (2000) Blood, 96:1425-1432; WO 00/34317; WO 98/52976), and direct chemical coupling of polyethylene glycol derivatives (Tsutsumi, et al., (2000) Proc. Natl. Acad. Sci. USA, 97:8548-8553). However, theses methods can be extremely time consuming, especially if considering multiple mutations simultaneously. While rational selection of surface residues can lead to decreased immunogenicity, some residue substitutions may be destablilizing and lead to poor folding. In addition, removing solvent exposed charged residues can be energetically unfavorable.

One way to overcome these problems is to use computational methods to design sequences that are more or less immunogenic relative to a target protein, but retain the structural properties to ensure proper folding and activity.

Accordingly, it is an object of the invention to use computational methods to screen for potential MHC, TCR, or BCR binding peptides. A wide variety of methods are known for generating and evaluating sequences. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)).

In particular, U.S. Ser. Nos. 60/061,097, 60/043,464, 60/054,678, 09/127,926 and PCT US98/07254 describe a method termed “Protein Design Automation”, or PDA, that utilizes a number of scoring functions to evaluate sequence stability.

Furthermore, it is an object of the present invention to provide computational methods for screening sequence libraries to select smaller libraries of protein sequences which can be made and evaluated for altered immunogenicity.

### SUMMARY OF THE INVENTION

In accordance with the objects outlined above, the present invention provides methods for modulating the immunogenicity of a target protein comprising the steps of inputting a protein backbone structure with variable residue positions into a computer, computationally generating a set of primary variant sequences, and applying a computational immunogenicity filter against the set of primary variant sequences to identify at least one candidate variant protein. The candidate protein is then made and tested to determine if the immunogenicity of the candidate protein is altered relative to the target protein.

The methods further comprise classifying each variable residue position as either a core, surface or boundary residue. The computationally generating step may include a Dead-End Elimination (DEE) computation or a Monte Carlo search. Generally, the primary variant sequences are optimized for at least one scoring function selected from the group consisting of Van der Waals potential scoring function, a hydrogen bond potential scoring function, an atomic solvation scoring function, a secondary structure propensity scoring function and an electrostatic scoring function.

In an additional aspect, the target protein is from a non human species and the candidate variant protein is rendered less immunogenic or non immunogenic in humans.

In an additional aspect, the present invention provides methods for modulating the immunogenicity of a target protein comprising the steps of inputting a protein backbone with variable residue positions into a computer, applying a computational immunogenicity filter to identify at least one candidate variant protein, computationally analyzing said variant protein for proper folding and stability, and generating a set of primary variant amino acid sequences.

### BRIEF DESCRIPTION OF THE DRAWINGS

FIG. 1 depicts the synthesis of a full-length gene and all possible mutations by PCR. Overlapping oligonucleotides corresponding to the full-length gene (black bar, Step 1) are synthesized, heated and annealed. Addition of Pfu DNA polymerase to the annealed oligonucleotides results in the 5′→3′ synthesis of DNA (Step 2) to produce longer DNA fragments (Step 3). Repeated cycles of heating, annealing (Step 4) results in the production of longer DNA, including some full-length molecules. These can be selected by a second round of PCR using primers (arrowed) corresponding to the end of the full-length gene (Step 5).

FIG. 2 depicts a preferred scheme for synthesizing a library of the invention. The wild-type gene, or any starting gene, such as the gene for the global minima gene, can be used. Oligonucleotides comprising different amino acids at the different variant positions can be used during PCR using standard primers. This generally requires fewer oligonucleotides and can result in fewer errors.

FIG. 3 depicts an overlapping extension method. At the top of FIG. 3 is the template DNA showing the locations of the regions to be mutated (black boxes) and the binding sites of the relevant primers (arrows). The primers R1 and R2 represent a pool of primers, each containing a different mutation; as described herein, this may be done using different ratios of primers if desired. The variant position is flanked by regions of homology sufficient to get hybridization. In this example, three separate PCR reactions are done for step 1. The first reaction contains the template plus oligos F1 and R1. The second reaction contains template plus F2 and R2, and the third contains the template and F3 and R3. The reaction products are shown. In Step 2, the products from Step 1 tube 1 and Step 1 tube 2 are taken. After purification away from the primers, these are added to a fresh PCR reaction together with F1 and R4. During the denaturation phase of the PCR, the overlapping regions anneal and the second strand is synthesized. The product is then amplified by the outside primers. In Step 3, the purified product from Step 2 is used in a third PCR reaction, together with the product of Step 1, tube 3 and the primers F1 and R3. The final product corresponds to the full length gene and contains the required mutations.

FIG. 4 depicts a ligation of PCR reaction products to synthesize the libraries of the invention. In this technique, the primers also contain an endonuclease restriction site (RE), either blunt, 5′ overhanging or 3′ overhanging. We set up three separate PCR reactions for Step 1. The first reaction contains the template plus oligos F1 and R1. The second reaction contains template plus F2 and R2, and the third contains the template and F3 and R3. The reaction products are shown. In Step 2, the products of step 1 are purified and then digested with the appropriate restriction endonuclease. The digestion products from Step 2, tube 1 and Step 2, tube 2 and ligate them togther with DNA ligase (step 3). The products are then amplified in Step 4 using primer F1 and R4. The whole process is then repeated by digesting the amplified products, ligating them to the digested products of Step 2, tube 3, and amplifying the final product by primers F1 and R3. It would also be possible to ligate all three PCR products from Step 1 together in one reaction, providing the two restriction sites (RET and RE2) were different.

FIG. 5 depicts blunt end ligation of PCR products. In this technique, the primers such as F1 and R1 do not overlap, but they abut. Again three separate PCR reactions are performed. The products from tube 1 and tube 2 are ligated, and then amplified with outside primers F1 and R4. This product is then .I gated with the product from Step 1, tube 3. The final products are then amplified with primers F1 and R3.

### DETAILED DESCRIPTION OF THE INVENTION

The present invention is directed to methods of using computational screening of protein sequence libraries (that can comprise up to 10⁸⁰ or more members) to select smaller libraries of protein sequences (that can comprise up to 10¹³ members) with altered immunogenicity. For example, if a protein with reduced immunogenicity is desired, a computational filter can be use to identify and replace residues known to elicit a immune response with compensatory residues that maintain the native fold and stability of the protein resulting in a protein that is non-immunogenic or less immunogenic than the starting protein.

Alternatively, it may be desirable to design proteins with increased immunogenicity. In this case, the computational filter can be applied to modify residues to introduce an antigenic motif to ensure proper folding and stability of the resultant protein.

In general, this can be done in one of two general ways. In a first embodiment, computational processing is used to generate a list of variant proteins that have an altered property such as stability. Then a computational filter is applied to select those variants with a high propensity for altered immunogenicity.

Alternatively, the computational filter is first applied to generate a list of variants with a propensity for altered immunogenicity, and then computational processing is done to select those variant that are likely to fold or to be stable.

In particular, a computational filter is used to screen for peptide fragments or amino acid residues that have the potential to bind to MHC class I and class II molecules, T cells and B cells. For example, databases for MHC ligands and peptide motifs can be searched and used to identify potential MHC class I or class II binding sequences (Rammensee, H., et al. (1999) Immunogenetics, 50:213-219). Computational methods are then used to structurally and chemically compensate for amino acid residues involved in binding to MHC molecules. For example, if a variant protein that is less immunogenic then the target protein is desired, computational methods can be used identify peptide sequences or amino acid residues predicted to elicit an immune response, replace these residues with residues predicted to be non immunogenic and then screen the resulting sequences for sequences that fold properly and are stable.

Rules for determining suitable replacements of antibody binding surface residues are emerging (see Meyer, D. L., et al. (2001) Protein Science, 10:491-503; Laroche, Y., (2000) Blood, 96:1425-1432; and Schwartz, H. L., (1999) J. Mol. Biol., 287:983-999). For example, aromatic surface residues are implicated in antigen-antibody binding. Replacement of aromatic surface residues such as tyrosine with smaller residues, such as serine, alanine or glycine can be done. Similarly, large patches of charged side chains can be replaced with small hydrophilic residues such as serine or alanine. Computational methods can then be applied to determine compensatory sequence changes to maintain the native fold and stability.

There are also some situations where it is desirable to increase the immunogenicity of a target protein. For example, activating populations of T cells toward a specific epitope has implications for controlling or eliminating viral pathogens or neoplasia. In this case, computational methods can be used to introduce T cell epitopes into less rigid, less structurally restricted loop regions of a target protein. Computational methods can then be used to modify the residues adjacent to the epitope insertion, ensuring energetic compatibility between the native protein and the grafted epitope.

Accordingly, the present invention provides methods for modulating the immunogenicity of a target protein. By “modulating” herein is meant that the immune response to a target protein is altered. That is, if a target protein elicits an immune response in a given species, the amino acid sequence of the target protein is changed such that the immune response is either reduced or enhanced. By “reduced” herein is meant that at least one immunological response is decreased relative to the wild-type protein. By “enhanced” herein is meant that at least one immunological response is increased relative to the wild-type protein. As will be recognized by those of skill in the art, not all identified sequences capable of eliciting a response need to be altered. For example, immune responses are generally not mounted against autologous circulating proteins, such as immunoglobulins and other serum proteins. Therefore, at least 5% of the sequences that are capable of eliciting a response are altered. Preferably at least 10% of the sequences are altered, more preferred is where at least 15% of the sequence are altered, even more preferred is when at least 20% of the sequences are altered, even more preferred is when at least 30% of the sequences are altered, even more preferred is when at least 40% of the sequences are altered, more preferred are where at least 50% of the sequences are altered, and most preferred is when 100% of the sequences are altered.

It should also be noted that altered immunogenicity is defined within a particular host organism. That is, in a preferred embodiment, target proteins (as defined below) are altered to exhibit altered immunogenicity within a human. Alternate host organisms include, bur are not limited to, rodents, (rats, mice, hamster, guinea pigs, etc.), primates, farm animals (including sheep, goats, pigs, cows, horses, etc.), and domestic animals, (including cats, dogs, rabbits, etc).

By “immunogenicity” herein refers to the ability of a protein to elicit an immune response. The ability of a protein to elicit an immune response depends on the amino acid sequence or sequences within the protein. Amino acid sequences capable of eliciting an immune response are referred to herein as “immunogenic sequences”. Preferably immunogenic sequences comprise “MHC binding sites”, “T cell epitopes” and “B cell epitopes” as outlined below.

As defined herein, the definition of immunogenicity is sufficiently broad to include the term “antigenicity”. “Antigenicity” refers to a the ability of a protein by itself to elicit an antibody response when recognized as a non-self molecule.

The response elicited by a protein with an immunogenic sequence involves both components of the immune system: the humoral component and the cellular component. Thus, “immune response” in the context of the invention includes any component of the humoral or cellular immune response.

Briefly, when a protein with immunogenic sequences is administered to a human, that protein is subjected to surveillance by both the humoral and cellular arms of the immune system. The immune system will respond to the protein if it is recognized as foreign and if the immune system is not already tolerant to the immunogenic sequence within the protein. For the humoral immune response, immature B cells displaying surface immunoglobulins (Igs) can bind to one or more sequences within the protein (B cell epitopes) if there is an affinity fit with the individual immunoglobulin and if the B cell epitope is exposed such that the Igs can access the B cell epitope. The process of Ig binding to the protein can, in the presence of suitable cytokines, stimulate the B cell to differentiate and divide to provide soluble forms of the original Ig which can complex with the protein to facilitate its clearance from an individual.

An effective B cell response also includes a parallel T cell response in order to provide the cytokines and other signals necessary to give rise to soluble antibodies. An effective T cell response requires the uptake of the of the protein or fragment thereof by antigen presenting cells (APCs); APCs include B cells or other cells such as macrophages, dendritic cells and other monocytes. The APCs then present the protein complexed with an MHC class II molecule at the cell surface. Such peptide-MHC II complexes can be recognized by helper T cells via the T cell receptor and this results in stimulation of the T cells and secretion of cytokines that provide help for B cells in their differentiation to antibody producing cells. As can be seen from the above discussion, an effective primary immune response to an immunogenic protein generally requires a combination of B and T cell responses to B and T cell specific sequences or epitopes.

Alternatively, if the immunogenic sequences are specific for MHC class I molecules, the MHC I antigen processing/presentation pathways are involved. MHC class I molecules gather fragments of proteins derived from infecting pathogens or “self” molecules and then display these fragments at the surface of an APC. The bound peptides are recognized by the TCRs of cytotoxic T lymphocytes and are the primary antigenic determinants of the cellular immune response. Thus, modulation of immunogenicity includes identifying peptides that stimulate T cell responses, termed T cell epitopes, changing the sequence of these peptides such that the cellular response to the protein is either reduced or enhanced. Additionally, modulation of immunogenicity also includes identifying peptides that stimulate B cell responses, termed “B cell epitopes” or “BCRs”, changing the sequence of these peptides such that the humoral response to the protein is altered. As will be understood by those of skill in the art, a single protein may contain both T and B cell epitopes, such that modification of both may alter both the humoral and cellular arms of the immune system.

In a preferred embodiment, the target protein is altered such that its MHC I response is altered. MHC class I molecules gather fragments of proteins derived from infecting viruses, intracellular parasites, or self molecules, either normally expressed or dysregulated by tumorigenesis, and then displays these molecular fragments at the cell surface. At the cell surface, the cell-bound MHC I-peptide complex exposed on the APC is displayed to T cells. The second characteristic of the MHC I molecule is the ability to interact with TCR which allows the APC bearing a particular MHC-peptide complex to engage an appropriate TCR. This is the first step in the activation of a cellular program leading to cytolysis of the APC as a target and/or the secretion of lymphokines by the T cell. The interaction with the TCR is dependent on both the peptide and the MHC molecule. MHC class I molecules show preferential restriction to CD8+ cells. An additional function of MHC class I molecules is to serve as elements for signal transduction to natural killer cells (Fundamental Immunology, fourth edition, W. E. Paul, ed., Lippincott-Raven Publishers, 1999, Chapter 8, pp 263-285).

In a preferred embodiment, the target protein is altered such that its MHC II response is altered. Exploiting similar molecular mechanisms to MHC class I molecules, MHC class II molecules bind peptides derived from the degradation of proteins ingested by MHC II expressing APCs, and displays them at the cell surface for recognition by specific T cells. The MHC II antigen presentation pathway is based on the initial assembly of the MHC II αβ heterodimer with a dual function molecule, the invariant chain (li) that serves as a chaperone to direct the αβ heterodimer to an endosomal, acidic protein processing location where it encounters antigenic peptides. The process of loading the MHC II molecule with antigenic peptide leads to the cell surface presentation of MHC II peptide complexes. MHC II recognizing T cells then secrete lymphokines and may be induced to proliferate. MHC class II molecules show preferential restriction to CD4+ cells. (Fundamental Immunology, fourth edition, W. E. Paul, ed., Lippincott-Raven Publishers, 1999, Chapter 8, pp 263-285).

In a preferred embodiment, the target protein is altered such that its TCR response is altered. TCRs occur as either of two distinct heterodimers, αβ or γδ, both of which are expressed with the non polymorphic CD3 polypeptides γ, δ, ε, ζ. The CD3 polypeptides, especially ζ and its variants, are critical for intracellular signaling. The αβ TCR heterodimer expressing cells predominate in most lymphoid compartments and are responsible for the classical helper or cytotoxic T cell responses. Im most cases, the αβ TCR ligand is a peptide antigen bound to a class I or a class II MHC molecule (Fundamental Immunology, fourth edition, W. E. Paul, ed., Lippincott-Raven Publishers, 1999, Chapter 10, pp 341-367).

In a preferred embodiment, the target protein is altered such that its BCR response is altered.

Antigen contact with a specific B cell triggers the transmembrane signaling function of the B cell antigen receptor (BCR). BCR molecules are rapidly internalized after antigen binding, leading to antigen uptake and degradation in endosomes or lysosomes. In the case of protein antigens, antigen-derived peptides bind in the groove of class II MHC molecules. Upon binding, this complex is sent to the cell surface, where it serves as a stimulus for specific helper T cells. Antigen recognition by the helper T cell induces it to form a tight and long lasting interaction with the B cell and to synthesize B cell growth and differentiation factors. B cells activated in this way may proliferate and terminally differentiate to antibody secreting cells (also called plasma cells) (Fundamental Immunology, fourth edition, W. E. Paul, ed., Lippincott-Raven Publishers, 1999, Chapters 6-7, pp 183-261)

Accordingly, the present invention is directed to methods for modulating the immunogenicity of a target protein. By “target protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acd. Sci. U.S.A. 89(20:9367-71 (1992)], generally depending on the method of synthesis. Thus “amino acid”, or “peptide residue”, as used herein means both naturally occurring and synthetic amino acids. For example, homo-phenylalanine, citrulline, and noreleucine are considered amino acids for the purposes of the invention. “Amino acid” also includes imino acid residues such as proline and hydroxyproline. In addition, any amino acid representing a component of the variant proteins of the present invention can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R- or the S-, depending upon its composition and chemical configuration. Such derivatives have the property of greatly increased stability, and therefore are advantageous in the formulation of compounds which may have longer in vivo half lives, when administered by oral, intravenous, intramuscular, intraperitoneal, topical, rectal, intraocular, or other routes.

In the preferred embodiment, the amino acids are in the (S) or L-configuration. If non-naturally occurring side chains are used, non-amino acid substituents may be used, for example to prevent or retard in vivo degradations. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218:U138-U138 Part 2 Aug. 22, 1999, both of which are expressly incorporated by reference herein.

Aromatic amino acids may be replaced with D- or L-naphylalanine, D- or L-Phenylglycine, D- or L-2-thieneylalanine, D- or L-1-, 2-, 3- or 4-pyreneylalanine, D- or L-3-thieneylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylphenylalanine, D- or L-p-methoxybiphenylphenylalanine, D- or L-2-indole(alkyl)alanines, and D- or L-alkylainines where alkyl may be substituted or unsubstituted methyl, ethyl, propyl, hexyl, butyl, pentyl, isopropyl, iso-butyl, sec-isotyl, iso-pentyl, non-acidic amino acids, of C1-C20.

Acidic amino acids can be substituted with non-carboxylate amino acids while maintaining a negative charge, and derivatives or analogs thereof, such as the non-limiting examples of (phosphono)alanine, glycine, leucine, isoleucine, threonine, or serine; or sulfated (e.g., —SO₃H) threonine, serine, or tyrosine.

Other substitutions may include unnatural hyroxylated amino acids may made by combining “alkyl” with any natural amino acid. The term “alkyl” as used herein refers to a branched or unbranched saturated hydrocarbon group of 1 to 24 carbon atoms, such as methyl, ethyl, n-propyl, isoptopyl, n-butyl, isobutyl, t-butyl, octyl, decyl, tetradecyl, hexadecyl, eicosyl, tetracisyl and the like. Alkyl includes heteroalkyl, with atoms of nitrogen, oxygen and sulfur. Preferred alkyl groups herein contain 1 to 12 carbon atoms. Basic amino acids may be substituted with alkyl groups at any position of the naturally occurring amino acids lysine, arginine, ornithine, citrulline, or (guanidino)-acetic acid, or other (guanidino)alkyl-acetic acids, where “alkyl” is define as above. Nitrile derivatives (e.g., containing the CN-moiety in place of COOH) may also be substituted for asparagine or glutamine, and methionine sulfoxide may be substituted for methionine. Methods of preparation of such peptide derivatives are well known to one skilled in the art.

In addition, any amide linkage in any of the variant polypeptides can be replaced by a ketomethylene moiety. Such derivatives are expected to have the property of increased stability to degradation by enzymes, and therefore possess advantages for the formulation of compounds which may have increased in vivo half lives, as administered by oral, intravenous, intramuscular, intraperitoneal, topical, rectal, intraocular, or other routes.

Additional amino acid modifications of amino acids of variant polypeptides of to the present invention may include the following: Cysteinyl residues may be reacted with alpha-haloacetates (and corresponding amines), such as 2-chloroacetic acid or chloroacetamide, to give carboxymethyl or carboxyamidomethyl derivatives. Cysteinyl residues may also be derivatized by reaction with compounds such as bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or ch loro-7-nitrobenzo-2-oxa-1 ,3-diazole.

Histidyl residues may be derivatized by reaction with compounds such as diethylprocarbonate e.g., at pH 5.5-7.0 because this agent is relatively specific for the histidyl side chain, and para-bromophenacyl bromide may also be used; e.g., where the reaction is preferably performed in 0.1M sodium cacodylate at pH 6.0.

Lysinyl and amino terminal residues may be reacted with compounds such as succinic or other carboxylic acid anhydrides. Derivatization with these agents is expected to have the effect of reversing the charge of the lysinyl residues.

Other suitable reagents for derivatizing alpha-amino-containing residues include compounds such as imidoesters/e.g., as methyl picolinimidate; pyridoxal phosphate; pyridoxal; chloroborohydride; trinitrobenzenesulfonic acid; 0-methylisourea; 2,4 pentanedione; and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin according to known method steps. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues per se is well-known, such as for introducing spectral labels into tyrosyl residues by reaction with aromatic diazonium compounds or tetranitromethane.

N-acetylimidizol and tetranitromethane may be used to form O-acetyl tyrosyl species and 3-nitro derivatives, respectively. Carboxyl side groups (aspartyl or glutamyl) may be selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Furthermore aspartyl and glutamyl residues may be converted to asparaginyl and glutaminyl residues by reaction with ammonium ions.

Glutaminyl and asparaginyl residues may be frequently deamidated to the corresponding glutamyl and aspartyl residues. Alternatively, these residues may be deamidated under mildly acidic conditions. Either form of these residues falls within the scope of the present invention.

The target protein may be any protein for which a three dimensional structure is known or can be generated; that is, for which there are three dimensional coordinates for each atom of the protein.

Generally this can be determined using X-ray crystallographic techniques, NMR techniques, de novo modeling, homology modeling, etc. In general, if X-ray structures are used, structures at 2A resolution or better are preferred, but not required.

The target proteins of the present invention may be from prokaryotes and eukaryotes, such as bacteria (including extremeophiles such as the archebacteria), fungi, insects, fish, and mammals. Suitable mammals include, but are not limited to, rodents (rats, mice, hamsters, guinea pigs, etc.), primates, farm animals (including sheep, goats, pigs, cows, horses, etc) and in the most preferred embodiment, from humans.

Thus, by “target protein” herein is meant a protein for which a library of variants, preferably with altered immunogenicity is desired. As will be appreciated by those in the art, any number of target proteins find use in the present invention. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides. In addition, protein variants, i.e. non-naturally occurring protein analog structures, may be used.

Suitable proteins include, but are not limited to, industrial, pharmaceutical, and agricultural proteins, including ligands, cell surface receptors, antigens, antibodies, cytokines, hormones, transcription factors, signaling modules, cytoskeletal proteins and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss-Prot enzyme database. Suitable protein backbones include, but are not limited to, all of those found in the protein data base compiled and serviced by the Research Collaboratory for Structural Bioinformatics (RCSB, formerly the Brookhaven National Lab).

Specifically, preferred pharmaceutical target proteins include, but are not limited to, those with known structures (including variants) including cytokines (IL-1 ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a; IFN-α-2B, TNF-α; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-lnhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Glial Cell-Derived Neurotrophic Factor, (as well as the 55 cytokines in PDB 1/12/99)); urokinase; Erythropoietin; other extracellular signalling moeities, including, but not limited to, hedgehog Sonic, hedgehog Desert, hedgehog Indian, hCG; coaguation factors including, but not limited to, TPA and Factor Vila; transcription factors, including but not limited to, p53, p53 tetramerization domain, Zn fingers (of which more than 12 have structures), homeodomains (of which 8 have structures), leucine zippers (of which 4 have structures); antibodies, including, but not limited to, cFv; viral proteins, including, but not limited to, hemagglutinin trimerization domain and hiv Gp41 ectodomain (fusion domain); intracellular signalling modules, including, but not limited to, SH2 domains (of which 8 structures are known), SH3 domains (of which 11 have structures), and Pleckstin Homology Domains; receptors, including, but not limited to, the extracellular Region Of Human Tissue Factor Cytokine-Binding Region Of Gp130, G-CSF receptor, erythropoietin receptor, Fibroblast Growth Factor receptor, TNF receptor, IL-1 receptor, IL-1 receptor/IL1ra complex, IL-4 receptor, INF-γ receptor alpha chain, MHC Class I, MHC Class II, T Cell Receptor, Insulin receptor, insulin receptor tyrosine kinase and human growth hormone receptor.

Specifically, preferred industrial target proteins include, but are not limited to, those with known structures (including variants) including proteases, (including, but not limited to papains, subtilisins), cellulases (including , but not limited to, endoglucanases I, II, and III, exoglucanases, xylanases, ligninases, cellobiohydrolases I, II, and III, carbohydrases (including, but not limited to glucoamylases, α-amylases, glucose isomerases) and lipases.

Specifically, preferred agricultural target proteins include, but are not limited to, those with known structures (including variants) including xylose isomerase, pectinases, cellulases, peroxidases, rubisco, ADP glucose phrophosphorlyase, as well as enzymes involved in oil biosynthesis, sterol biosynthesis, carbohydrate biosynthesis, and the synthesis of secondary metabolites.

In a preferred embodiment, the methods of the invention involve starting with a target protein and using computational analysis to generate a set of primary sequences. There are a wide variety of computational methods that can be used including sequence alignments of related proteins, structural alignments, structural prediction models, databases, or (preferably) protein design automation computational analysis. Similarly, libraries of primary variant sequences can be generated via sequence screening using a set of scaffold structures that are created by perturbing the starting structure (using any number of techniques such as molecular dynamics, Monte Carlo analysis) to make changes to the protein (including backbone and sidechain torsion angle changes). Optimal sequences can be selected for each starting structures (or, some set of the top sequences) to make libraries of primary variant sequences.

Some of these techniques result in the list of sequences in the primary library being “scored”, or “ranked” on the basis of some particular criteria. In some embodiments, lists of sequences that are generated without ranking can then be ranked using techniques as outlined below.

Generally, there are a variety of computational methods that can be used to generate a library of primary variant sequences. In a preferred embodiment, sequence based methods are used. Alternatively, structure based methods, such as PDA, described in detail below, are used. Other models for assessing the relative energies of sequences with high precision include Warshel, Computer Modeling of Chemical Reactions in Enzymes and Solutions, Wiley & Sons, New York, (1991), hereby expressly incorporated by reference.

Similarly, molecular dynamics calculations can be used to computationally screen sequences by individually calculating mutant sequence scores and compiling a rank ordered list.

In a preferred embodiment, residue pair potentials can be used to score sequences (Miyazawa et al., Macromolecules 18(3):534-552 (1985), expressly incorporated by reference) during computational screening.

In a preferred embodiment, sequence profile scores (Bowie et al., Science 253(5016):164-70 (1991), incorporated by reference) and/or potentials of mean force (Hendlich et al., J. Mol. Biol. 216(1):167-180 (1990), also incorporated by reference) can also be calculated to score sequences. These methods assess the match between a sequence and a 3D protein structure and hence can act to screen for fidelity to the protein structure. By using different scoring functions to rank sequences, different regions of sequence space can be sampled in the computational screen.

Furthermore, scoring functions can be used to screen for sequences that would create metal or co-factor binding sites in the protein (Hellinga, Fold Des. 3(1):R1-8 (1998), hereby expressly incorporated by reference). Similarly, scoring functions can be used to screen for sequences that would create disulfide bonds in the protein. These potentials attempt to specifically modify a protein structure to introduce a new structural motif.

In a preferred embodiment, sequence and/or structural alignment programs can be used to generate primary libraries. As is known in the art, there are a number of sequence-based alignment programs;

including for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise.

The source of the sequences can vary widely, and include taking sequences from one or more of the known databases, including, but not limited to, SCOP (Hubbard, et al., Nucleic Acids Res 27(1):254-256. (1999)); PFAM (Bateman, et al., Nucleic Acids Res 27(1):260-262. (1999)); VAST (Gibrat, et al., Curr Opin Struct Biol 6(3):377-385. (1996)); CATH (Orengo, et al., Structure 5(8):1093-1108. (1997)); PhD Predictor (http://www.embl-heidelberg.de/predictprotein/predictprotein.html); Prosite (Hofmann, et al., Nucleic Acids Res 27(1):215-219. (1999)); PIR (http://www.mips.biochem.mpq.de/proj/protseqdb/): Gen Bank (httD://www.ncbi.nlm.nih.gov/); PDB (www.rcsb.org) and BIND (Bader, et al., Nucleic Acids Res 29(1):242-245. (2001)).

In addition, sequences from these databases can be subjected to continguous analysis or gene prediction; see Wheeler, et al., Nucleic Acids Res 28(1):10-14. (2000) and Burge and Karlin, J Mol Biol 268(1):78-94. (1997).

As is known in the art, there are a number of sequence alignment methodologies that can be used.

For example, sequence homology based alignment methods can be used to create sequence alignments of proteins related to the target structure (Altschul et al., J. Mol. Biol. 215(3):403 (1990), incorporated by reference). These sequence alignments are then examined to determine the observed sequence variations. These sequence variations are tabulated to define a primary library. In addition, as is further outlined below, these methods can also be used to generate secondary libraries.

Sequence based alignments can be used in a variety of ways. For example, a number of related proteins can be aligned, as is known in the art, and the “variable” and “conserved” residues defined; that is, the residues that vary or remain identical between the family members can be defined. These results can be used to generate a probability table, as outlined below. Similarly, these sequence variations can be tabulated and a secondary library defined from them as defined below. Alternatively, the allowed sequence variations can be used to define the amino acids considered at each position during the computational screening. Another variation is to bias the score for amino acids that occur in the sequence alignment, thereby increasing the likelihood that they are found during computational screening but still allowing consideration of other amino acids. This bias would result in a focused primary library but would not eliminate from consideration amino acids not found in the alignment. In addition, a number of other types of bias may be introduced. For example, diversity may be forced; that is, a “conserved” residue is chosen and altered to force diversity on the protein and thus sample a greater portion of the sequence space. Alternatively, the positions of high variability between family members (i.e. low conservation) can be randomized, either using all or a subset of amino acids. Similarly, outlier residues, either positional outliers or side chain outliers, may be eliminated.

Similarly, structural alignment of structurally related proteins can be done to generate sequence alignments. There are a wide variety of such structural alignment programs known. See for example VAST from the NCBI (http://www.ncbi.nlm.nih.gov:80/StructureNAST/vast.shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9):739-747. (1998)); (Orengo et al., Structure 5(8):1093-108 (1997); Dali (Holm et al., Nucleic Acid Res. 26(1):316-9 (1998), all of which are incorporated by reference). These structurally-generated sequence alignments can then be examined to determine the observed sequence variations.

Libraries of primary variant sequences can be generated by predicting secondary structure from sequence, and then selecting sequences that are compatible with the predicted secondary structure. There are a number of secondary structure prediction methods, including, but not limited to, threading (Bryant and Altschul, Curr Opin Struct Biol 5(2):236-244. (1995)), Profile 3D (Bowie, et al., Methods Enzymol 266(598-616 (1996); MONSSTER (Skolnick, et al., J Mol Biol 265(2):217-241. (1997); Rosetta (Simons, et al., Proteins 37(S3):171-176 (1999); PSI-BLAST (Altschul and Koonin, Trends Biochem Sci 23(11):444-447. (1998)); Impala (Schaffer, et al., Bioinformatics 15(12):1000-1011. (1999)); HMMER (McClure, et al., Proc Int Conf Intell Syst Mol Biol 4(155-164 (1996)); Clustal W (http://www.ebi.ac.uk/clustalw/); BLAST (Altschul, et al., J Mol Biol 215(3):403-410. (1990)), helix-coil transition theory (Munoz and Serrano, Biopolymers 41:495, 1997), neural networks, local structure alignment and others (e.g., see in Selbig et al., Bioinformatics 15:1039, 1999).

Similarly, as outlined above, other computational methods are known, including, but not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994); PROSA (Heindlich et al., J. Mol. Biol. 216:167-180 (1990); THREADER (Jones et al., Nature 358:86-89 (1992), and other inverse folding methods such as those described by Simons et al. (Proteins, 34:535-543, 1999), Levitt and Gerstein (PNAS USA, 95:5913-5920,1998), Godzik et al., PNAS, V89, PP 12098-102; Godzik and Skolnick (PNAS USA, 89:12098-102, 1992), Godzik et al. (J. Mol. Biol. 227:227-38, 1992) and two profile methods (Gribskov et al. PNAS 84:4355-4358 (1987) and Fischer and Eisenberg, Protein Sci. 5:947-955 (1996), Rice and Eisenberg J. Mol. Biol. 267:1026-1038(1997)), all of which are expressly incorporated by reference. In addition, other computational methods such as those described by Koehl and Levitt (J. Mol. Biol. 293:1161-1181 (1999); J. Mol. Biol. 293:1183-1193 (1999); expressly incorporated by reference) can be used to create a protein sequence library which can optionally then be used to generate a smaller secondary library for use in experimental screening for improved properties and function.

In addition, there are computational methods based on forcefield calculations such as SCMF that can be used as well for SCMF, see Delarue et la. Pac. Symp. Biocomput. 109-21 (1997), Koehl et al., J.

Mol. Biol. 239:249 (1994); Koehl et al., Nat. Struc. Biol. 2:163 (1995); Koehl et al., Curr. Opin. Struct. 15-Biol. 6:222 (1996); Koehl et al., J. Mol. Bio. 293:1183 (1999); Koehl et al., J. Mol. Biol. 293:1161 (1999); Lee J. Mol. Biol. 236:918 (1994); and Vasquez Biopolymers 36:53-70 (1995); all of which are expressly incorporated by reference. Other forcefield calculations that can be used to optimize the conformation of a sequence within a computational method, or to generate de novo optimized sequences as outlined herein include, but are not limited to, OPLS-AA (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem May 1994;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al.,(1988) Proteins: Structure, Function and Genetics, v4,pp3l-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference. In fact, as is outlined below, these forcefield methods may be used to generate the secondary library directly; that is, no primary library is generated; rather, these methods can be used to generate a probability table from which the secondary library is directly generated, for example by using these forcefields during an SCMF calculation.

In a preferred embodiment, the computational method used to generate the primary library is Protein Design Automation™ (PDA™) technology, as is described in U.S. Ser. Nos. 60/061,097, 60/043,464, 60/054,678, 09/127,926, 09/782,004 and PCT US98/07254, all of which are expressly incorporated herein by reference. Briefly, PDA can be described as follows: A known protein structure is used as the starting point. The residues to be optimized are then identified, which may be the entire sequence or subset(s) thereof. The side chains of any positions to be varied are then removed. The resulting structure consisting of the protein backbone and the remaining sidechains is called the template. Each variable residue position is then preferably classified as a core residue, a surface residue, or a boundary residue; each classification defines a subset of possible amino acid residues for the position (for example, core residues generally will be selected from the set of hydrophobic residues, surface residues generally will be selected from the hydrophilic residues, and boundary residues may be either). Each amino acid can be represented by a discrete set of all allowed conformers of each side chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

Two sets of interactions are then calculated for each rotamer at every position: the interaction of the rotamer side chain with all or part of the backbone (the “singles” energy, also called the rotamer/template or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably.

DEE can be rerun comparing pairs of rotamers, or combinations of rotamers, which will eventually result in the determination of a single sequence which represents the global optimum energy.

Once the global solution has been found, a Monte Carlo search may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated.

Monte Carlo searching is a sampling technique to explore sequence space around the global minimum or to find new local minima distant in sequence space. As is more additionally outlined below, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Similarly, for all the sampling techniques, the acceptance criteria of whether a sampling jump is accepted can be altered.

As outlined in U.S. Ser. No. 09/127,926, the protein backbone (comprising (for a naturally occurring protein) the nitrogen, the carbonyl carbon, the α-carbon, and the carbonyl oxygen, along with the direction of the vector from the α-carbon to the β-carbon) may be altered prior to the computational analysis, by varying a set of parameters called supersecondary structure parameters.

Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by doing a number of steps of conjugate gradient minimization (Mayo et al., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at it's N-terminus is said to have a methionine at residue or amino acid position 1, with the next residues as 2, 3, 4, etc. At each position, the wild type (i.e. naturally occurring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein which may be designed this way, there is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid.

Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In a preferred embodiment, residues which can be fixed include, but are not limited to, structurally or biologically functional residues; alternatively, biologically functional residues may specifically not be fixed. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

In a preferred embodiment, each variable position is classified as either a core, surface or boundary residue position, although in some cases, as explained below, the variable position may be set to glycine to minimize backbone strain. In addition, as outlined herein, residues need not be classified, they can be chosen as variable and any set of amino acids may be used. Any combination of core, surface and boundary positions can be utilized: core, surface and boundary residues; core and surface residues; core and boundary residues, and surface and boundary residues, as well as core residues alone, surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modelling. Alternatively, a preferred embodiment utilizes an assessment of the orientation of the Cα-Cβ vectors relative to a solvent accessible surface computed using only the template Ca atoms, as outlined in U.S. Ser. Nos. 60/061,097, 60/043,464, 60/054,678, 09/127,926 and PCT US98/07254. Alternatively, a surface area calculation can be done.

Once each variable position is classified as either core, surface or boundary, a set of amino acid side chains, and thus a set of rotamers, is assigned to each position. That is, the set of possible amino acid side chains that the program will allow to be considered at any particular position is chosen. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the a scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be). Additionally, in some preferred embodiments, a set of 18 naturally occuring amino acids (all except cysteine and proline, which are known to be particularly disruptive) are used.

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins which dimerize or multimerize, or have ligand binding sites, may contain hydrophobic surface residues, etc. In addition, residues that do not allow helix “capping” or the favorable interaction with an α-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a φ angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds as outlined in U.S. Ser. No. 09/127,926 and PCT US98/07254. This processing step entails analyzing interactions of the rotamers with each other and with the protein backbone to generate optimized protein sequences. Simplistically, the processing initially comprises the use of a number of scoring functions to calculate energies of interactions of the rotamers, either to the backbone itself or other rotamers. Preferred PDA™ technology scoring functions include, but are not limited to, a Van der Waals potential scoring function, a hydrogen bond potential scoring function, an atomic solvation scoring function, a secondary structure propensity scoring function and an electrostatic scoring function. As is further described below, at least one scoring function is used to score each position, although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:

Eₜₒₜₐₗ=nE_{vdw}+nEₐₛ+nE_{h-bonding}+nEₛₛ+nE_{elec} Equation 1

In Equation 1, the total energy is the sum of the energy of the van der Waals potential (EVdW), the energy of atomic solvation (E_{as), the energy of hydrogen bonding (E}_{h-bonding}), the energy of secondary structure (Eₛₛ) and the energy of electrostatic interaction (E_{elec}). The term n is either 0 or 1, depending on whether the term is to be considered for the particular residue position.

As outlined in U.S. Ser. Nos. 60/061,097, 60/043,464, 60/054,678, 09/127,926 and PCT US98/07254, any combination of these scoring functions, either alone or in combination, may be used. Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered. The term “portion”, as used herein, with regard to a protein refers to a fragment of that protein. This fragment may range in size from 10 amino acid residues to the entire amino acid sequence minus one amino acid. Accordingly, the term “portion”, as used herein, with regard to a nucleic refers to a fragment of that nucleic acid. This fragment may range in size from 10 nucleotides to the entire nucleic acid sequence minus one nucleotide.

In a preferred embodiment, the first step of the computational processing is done by calculating two sets of interactions for each rotamer at every position: the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the EHB is calculated for each possible rotamer at every variable position. Similarly, for the van der Waals scoring function, every atom of the rotamer is compared to every atom of the template (generally excluding the backbone atoms of its own residue), and the E_{vdW} is calculated for each possible rotamer at every variable residue position. In addition, generally no van der Waals energy is calculated if the atoms are connected by three bonds or less. For the atomic solvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdW} is calculated for each possible rotamer pair at every two variable residue positions. For the atomic salvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

In addition, as will be appreciated by those in the art, a variety of force fields can be used in the PDA™ technology calculations, including, but not limited to, Dreiding I and Dreiding II (Mayo et al, J. Phys. Chem. 948897 (1990)), AMBER (Weiner et al., J. Amer. Chem. Soc. 106:765 (1984) and Weiner et al., J. Comp. Chem. 106:230 (1986)), MM2 (Allinger J. Chem. Soc. 99:8127 (1977), Liljefors et al., J. Com. Chem. 8:1051 (1987)); MMP2 (Sprague et al., J. Comp. Chem. 8:581 (1987)); CHARMM (Brooks et al., J. Comp. Chem. 106:187 (1983)); GROMOS; and MM3 (Allinger et al., J. Amer. Chem. Soc. 111:8551 (1989)), OPLS-AA (Jorgensen, et al., J. Am. Chem. Soc. (1996), v 118, pp 11225-11236; Jorgensen, W. L.; BOSS, Version 4.1; Yale University: New Haven, CT (1999)); OPLS (Jorgensen, et al., J. Am. Chem. Soc. (1988), v 110, pp 1657ff; Jorgensen, et al., J Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem May 1994;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al.,(1988) Proteins: Structure, Function and Genetics, v4,pp3147); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

Once the singles and doubles energies are calculated and stored, the next step of the computational processing may occur. As outlined in U.S. Ser. No. 09/127,926 and PCT US98/07254, preferred embodiments utilize a Dead End Elimination (DEE) step, and preferably a Monte Carlo step.

PDA™ technology, viewed broadly, has three components that may be varied to alter the output (e.g. the primary library): the scoring functions used in the process; the filtering technique, and the sampling technique.

In a preferred embodiment, the scoring functions may be altered. In a preferred embodiment, the scoring functions outlined above may be biased or weighted in a variety of ways. For example, a bias towards or away from a reference sequence or family of sequences can be done; for example, a bias towards wild-type or homolog residues may be used. Similarly, the entire protein or a fragment of it may be biased; for example, the active site may be biased towards wild-type residues, or domain residues towards a particular desired physical property can be done. Furthermore, a bias towards or against increased energy can be generated. Additional scoring function biases include, but are not limited to applying electrostatic potential gradients or hydrophobicity gradients, adding a substrate or binding partner to the calculation, or biasing towards a desired charge or hydrophobicity.

In addition, in an alternative embodiment, there are a variety of additional scoring functions that may be used. Additional scoring functions include, but are not limited to torsional potentials, or residue pair potentials, or residue entropy potentials. Such additional scoring functions can be used alone, or as functions for processing the library after it is scored initially.

In a preferred embodiment, a variety of process filtering techniques can be done, including, but not limited to, DEE and its related counterparts. Additional filtering techniques include, but are not limited to branch-and-bound techniques for finding optimal sequences (Gordon and Majo, Structure Fold. Des. 7:1089-98, 1999), and exhaustive enumeration of sequences. It should be noted however, that some techniques may also be done without any filtering techniques; for example, sampling techniques can be used to find good sequences, in the absence of filtering.

As will be appreciated by those in the art, once an optimized sequence or set of sequences is generated, (or again, these need not be optimized or ordered) a variety of sequence space sampling methods can be done, either in addition to the preferred Monte Carlo methods, or instead of a Monte Carlo search. That is, once a sequence or set of sequences is generated, preferred methods utilize sampling techniques to allow the generation of additional, related sequences for testing.

These sampling methods can include the use of amino acid substitutions, insertions or deletions, or recombinations of one or more sequences. As outlined herein, a preferred embodiment utilizes a Monte Carlo search, which is a series of biased, systematic, or random jumps. However, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Jumps where multiple residue positions are coupled (two residues always change together, or never change together), jumps where whole sets of residues change to other sequences (e.g., recombination). Similarly, for all the sampling techniques, the acceptance criteria of whether a sampling jump is accepted can be altered, to allow broad searches at high temperature and narrow searches close to local optima at low temperatures. See Metropolis et al., J. Chem Phys v21, pp 1087, 1953, hereby expressly incorporated by reference.

In addition, it should be noted that the preferred methods of the invention result in a rank ordered list of sequences; that is, the sequences are ranked on the basis of some objective criteria. However, as outlined herein, it is possible to create a set of non-ordered sequences, for example by generating a probability table directly (for example using SCMF analysis or sequence alignment techniques) that lists sequences without ranking them. The sampling techniques outlined herein can be used in either situation.

In a preferred embodiment, Boltzman sampling is done. As will be appreciated by those in the art, the temperature criteria for Boltzman sampling can be altered to allow broad searches at high temperature and narrow searches close to local optima at low temperatures (see e.g., Metropolis et al., J. Chem. Phys. 21:1087, 1953).

In a preferred embodiment, the sampling technique utilizes genetic algorithms, e.g., such as those described by Holland (Adaptation in Natural and Artifical Systems, 1975, Ann Arbor, U. Michigan Press). Genetic algorithm analysis generally takes generated sequences and recombines them computationally, similar to a nucleic acid recombination event, in a manner similar to “gene shuffling”. Thus the “jumps” of genetic algorithm analysis generally are multiple position jumps. In addition, as outlined below, correlated multiple jumps may also be done. Such jumps can occur with different crossover positions and more than one recombination at a time, and can involve recombination of two or more sequences. Furthermore, deletions or insertions (random or biased) can be done. In addition, as outlined below, genetic algorithm analysis may also be used after the secondary library has been generated.

In a preferred embodiment, the sampling technique utilizes simulated annealing, e.g., such as described by Kirkpatrick et al. (Science, 220:671-680, 1983). Simulated annealing alters the cutoff for accepting good or bad jumps by altering the temperature. That is, the stringency of the cutoff is altered by altering the temperature. This allows broad searches at high temperature to new areas of sequence space, altering with narrow searches at low temperature to explore regions in detail.

In addition, as outlined below, these sampling methods can be used to further process a secondary library to generate additional secondary libraries (sometimes referred to herein as tertiary libraries).

Thus, the primary library can be generated in a variety of computational ways, including structure based methods such as PDA™, or sequence based methods, or combinations as outlined herein.

The computational processing results in a set of optimized variant candidate sequences. Optimized variant candidate protein sequences are generally different from the target protein sequence in regions critical for MHC, TCR or BCR binding. Preferably, each optimized variant candidate sequence comprises at least about 1 variant amino acid from the starting or target sequence, with 3-5 being preferred. Preferably, the variant residues are located in noncontiguous regions.

Accordingly, in a preferred embodiment, the present invention is directed to methods of computationally processing a target protein, or fragment thereof, to produce a variant candidates protein or a set of variant candidates protein sequences.

Thus, in a preferred embodiment, the variant candidate proteins of the invention have an amino acid sequence that differs from the target protein in at least one MHC, TCR, or BCR binding site. Preferably, if a less immunogenic protein is desired, the candidate variant protein differs from the target protein by the elimination of at least one MHC, TCR, or BCR binding site. Alternatively, if a more immunogenic protein is desired, the candidate variant protein differs from the target protein via the addition of at least one MHC, TCR, or BCR binding site.

Accordingly, the computational processing results in a set of primary variant sequences, that may be optimized protein sequences if some sort of ranking or scoring functions are used. These optimized protein sequences are generally, but not always, significantly different from the target sequence from which the backbone was taken. That is, each optimized protein sequence preferably comprises at least about 5-10% variant amino acids from the starting target or wild-type sequence, with at least about 15-20% changes being preferred and at least about 30% changes being particularly preferred.

In a preferred embodiment, a computational immunogenicity filter is applied to the set of primary library sequences. By “computational immunogenicity filter” herein is meant a any one of a number of scoring functions derived from data on binding of peptides to MHC molecules, or T cell epitopes or B cell epitopes. These scoring functions are used to rescore the set of primary library sequences to eliminate potentially immunogenic sequences, or eliminate non-immunogenic sequences. PDA will then be used to structurally and chemically compensate for any residues, including surface residues, removed or added to modulate immunogenicity.

In a preferred embodiment, PDA™ technology will be used to structurally and chemically compensate for either the removal or addition of amino acid residues encoding linear epitopes displayed by MHC class I and II molecules that are recognized by TCRs.

In a preferred embodiment, PDA™ technology will be used to structurally and chemically compensate for either the removal or addition of amino acid residues encoding conformational epitopes, that are sensed by membrane bound antibodies on naive B cells.

In other embodiments, the computational immunogenicity filter is applied before ir during the computational generation of a set of primary sequences. Using this approach, a set of primary sequences is generated that potentially either lack or include immunogenic sequences. PDA™ technology is then run on these sequences to identify those sequences that retain the native fold and are at least as stable as the starting target protein.

The current understanding of the rules for peptide selection by MHC molecules is derived from sequencing of peptides and natural peptide libraries extracted from MHC proteins, from analyses of the effects of mutations in sequences of unknown CTL epitopes on peptide binding to MHC molecules and on T cell responses, as well as from crystal structure analyses and molecular dynamic studies of defined MHC-peptide complexes (Meister, G. E., et al. (1995) Vaccine, 13:581-591; Malios, R. R., (1999) Bioinformatics Savoie, C. J. et al. (1999) Pac Symp Biocomput., 182-9; Brusic, V., et al., (1998) Bioinformatics, Mallios, R. R., (1998) J. Comp. Biol., 5:703-711; Altuvia, Y., et al. (1997) Human Immunology, 58:1-11; Udaka, et al., (1995) J. Exp. Med., 181:2097-2108; Hammer, J. et al. (1994) Behring. Inst. Mitt. 94:124-132). In addition, databases consisting of thousands of peptide sequences know to bind MHC molecules have been compiled (Buus, supra) and several techniques have been developed to analyze sequences of full length proteins to predict the presence of potentially immunogenic sequences (Hiemstra, H. S. et al. (2000) Curr. Op. Immunol., 12:80-84; Malios, R. R., (1999) Bioinformatics, 15:432-439; Sturniolo, T., et al. (1999) Nature Biotechnology, 17:555-561; Brusic, V., et al., (1998) Bioinformatics, 14:121-130; Mallios, R. R., (1998) J Comp. Biol., 5:703-711; Shastri, N. (1996) Curr. Op. Immunol., 8:271-277; Hammer, J. (1995) Curr. Op. Immunol., 7:263-269; Meister, G. E., et al. (1995) Vaccine, 13:581-591; Udaka, K., et al. (1995) J Exp. Med., 181:20972108; Hammer, J. et al. (1994) Behring. Inst. Mitt. 94:124-132; Hammer, J., et al. (1994) J. Exp. Med., 180: 2353-2358; and, Rudenshky, A. Y., et al. (1991) Nature, 353:622-627; all of which are expressly incorporated herein by reference).

In a preferred embodiment, primary variant sequences are screened for peptide fragments potentially capable of binding to MHC class I molecules. The MHC I ligands are mostly octa-or nonapeptides and show MHC allele specific sequence motifs as determined by pool sequencing of natural isolates.

Crystal structure analysis has identified a peptide binding cleft, i.e., groove, framed by two a helices and a β pleated sheet. The cleft is stabilized from beneath by the noncovalently associated β2 microglobulin. Specific pockets in the binding groove accommodate the anchor residues of the peptide. The orientation of the peptides is determined by conserved side chains of the MHC I protein that compensate the NH₂₋ and COOH-terminal charges.

A given MHC class I peptide binding groove can bind hundreds or thousands of different peptides, identical or homologous at only a few side chain positions. Comparisons of the structures of numerous class I peptide-MHC complexes reveals that this flexibility is achieved by the structurally equivalent binding of a small subset of each peptide's residues. Among these, the binding of charged and polar atoms of the peptide main chain provides essential side-chain-independent peptide MHC interactions. This collection of hydrogen bonds and van der Waals contacts helps to stabilize the binding of any peptide capable of adopting the required backbone conformation. Additional interactions with a few peptide side chains supplement the main-chain binding energy and impose some sequence selectivity on the peptides bound by a particular MHC molecule (Madden, D. R. (1995) Annu. Rev. Immunol., 13:587-622). Rules for identifying MHC I binding sites have been described inAltuvia, Y., et al (1997) Human Immunology, 58:1-11; and, Meister, GE., et al (1995) Vaccine: 6:581-591; hereby incorporated by reference in their entirety).

In a preferred embodiment, potential MHC class I binding sites will be replaced with amino acid residues which structurally and chemically compensate for the anchor residues removed to reduce or eliminate peptide binding to MHC class I molecules. Preferably, potential MHC I binding motifs will be identified by matching a database of published motifs, such as SYFPEITHI (Rammensee, H., et al., (1999) Immunogenetics, 50:213-219; http://134.2.96.221/scripts/MHCServer.dll/home.html)); http://wehih.wehi.edu.au/mhcpep/.

In additional embodiments, non-anchoring residues will be eliminated.

In a preferred embodiment, primary variant sequences will be screened for peptide fragments predicted to bind to MHC class II molecules. Class II ligands consist of 12 to 25 amino acids, nine of which occupy the binding groove; between two and four are anchored in the pockets. As in the class I ligands, the nonanchoring amino acids play a secondary, but still significant role (Rammensee, H., et al., (1999) Immunogenetics, 50:213-219). Rules for identifying MHC II binding sites have been described in Hammer, J. et al., (1994) Behring. Inst. Mitt., 94: 124-132; Hammer, J. et al., (1995) J. Exp. Med., 180:2353-2358; Mallios, R. R. (1998) J. Com. Biol., 5:703-711; Brusic, V., et al., (1998) Bioinformatics, 14:121-130; Mallios, R. R. (1999) Bioinformatics, 15:432-439; hereby incorporated by reference in their entirety).

In a preferred embodiment, potential MHC class II binding sites will be replaced with amino acid residues which structurally and chemically compensate for anchor residues removed to eliminate MHC I binding sites. Preferably, potential MHC I binding sites will be identified by matching a database of published motifs, such as SYFPEITHI (Rammensee, H., et al., (1999) Immunogenetics, 50:213-219; http://134.2.96.221/scripts/MHCServer.dll/home.html) or http://wehih.wehi.edu.au/mhcpep/). Alternatively, the prediction of binding to class II molecules will use the method of virtual matrices as described by Sturniolo, T, et al. (1999) Nature Biotechnology, 17:555-561).

In additional embodiments, non-anchoring residues will be eliminated.

In a preferred embodiment, only sequences altered by the computational methods described herein are considered.

In other embodiments, peptide sequences present in autologous proteins (i.e., circulating human proteins such as immunoglobulins, albumin, etc.) are ignored.

In a preferred embodiment, primary variant sequences will be screened for peptide fragments predicted to function as T cell epitopes. In a preferred embodiment, potential T cell epitopes will be replaced with amino acid residues which structurally and chemically compensate for the residues removed to eliminate the T cell epitope. Preferably, potential T cell epitopes will be identified by matching a database of published motifs (Walden, P., (1996) Curr. Op. Immunol., 8:68-74). Other methods of identifying T cell epitopes which are useful in the present invention include those described by Hemmer, B., et al. (1998) J. Immunol., 160:3631-3636; Walden, P., et al. (1995) Biochemical Society Transactions, 23; Anderton, S. M., et al., (1999) Eur. J. Immunol., 29:1850-1857; Correia-Neves, M., et al., (1999) J. Immunol., 163:5471-5477; Shastri, N., (1995) Curr. Op. Immunol., 7:258-262; Hiemstra, H. S., (2000) Curr. Op. Immunol., 12:80-84; and Meister, G. E., et al., (1995) Vaccine, 13:581-591; all of which are hereby expressly incorporated by reference in their entirety).

In other embodiments, T cell epitopes will be introduced into primary sequence libraries in regions that will not affect the native folding and stability of the target protein. T cell epitopes will be selected from databases of known MHC I binding peptides, MHC II binding peptides, and T cell epitopes as described above.

In a preferred embodiment, primary variant sequences will be screened for peptide fragments predicted to bind to antibodies. In a preferred embodiment, potential B cell epitopes will be replaced with smaller neutral residues to reduce the immunogenicity of the sequence as described by Meyer et al. (Meyer, D. L., et al. (2001), Protein Sci., 10:491-503; see also Schwartz, H L., et al. (1999) J. Mol Biol. 287:983-999; and Laroche, Y., et al., (2000) Blood, 96:1425-1432).

In other embodiments, B cell epitopes will be introduced into primary sequence libraries in regions that will not affect the native folding and stability of the target protein. In particular, charged, aromatic, or large hydrophobic residues on the surface of the target protein are added.

In a preferred embodiment, at least one candidate variant protein is identified in which at least one sequence capable of interacting with an MHC class I or class II molecule, a TCR or BCR has been altered. Any method of identifying potential or actual MHC, TCR or BCR sequences can be used in the invention. Acceptable methods include computational or physical methods. Acceptable computational methods include the use of algorithms such as OptiMer and EpiMer (Meister, GE., et al. (1995) Vaccine, 6:581-591); iterative stepwise discriminant analysis metal algorithm (Mallios, RR., (1999) Bioinformatics, 15:432-439);and structure based (Altuvia, Y., (1997) Human Immunology 58:1-11 and predictive methods combining an evolutionary algorithm and artificial neural network (Brusic, V., et al. (1998) Bioinformatics, 14:121-130), virtual matrices (Sturniolo, T., et al. (1999) Nature Biotechnology, 17:555-561) and BONSAI decision trees (Savoie, CJ., et al (1999) Pac Symp Biocomput., 182-9).

Acceptable physical methods include high affinity binding assays (Hammer, J., et al. (1993) Proc. Natl. Acad. Sci. USA, 91:4456-4460; Sarobe, P. et al. (1998) J. Clin. Invest., 102:1239-1248), T cell proliferation and CTL assays (Hemmer, B., et al., (1998) J. Immunol., 160:3631-3636).

Having identified potential MHC, TCR, or BCR sequences, these sequences are then modified by the replacement of one or more amino acids as described below. Once the candidate variant protein has been so modified, the protein is then tested to determine if its activity is similar to the target protein. The variant may retain full activity, or retain a sufficient proportion of its activity to be useful.

The variant proteins and nucleic acids of the invention are distinguishable from the naturally occurring target protein. By “naturally occurring” or “wild type” or grammatical equivalents, herein is meant an amino acid sequence or a nucleotide sequence that is found in nature and includes allelic variations; that is, an amino acid sequence or a nucleotide sequence that usually has not been intentionally modified. Accordingly, by “non-naturally occurring” or “synthetic” or “recombinant” or grammatical equivalents thereof, herein is meant an amino acid sequence or a nucleotide sequence that is not found in nature; that is, an amino acid sequence or a nucleotide sequence that usually has been intentionally modified. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purpose of the invention. Thus, the variant proteins and nucleic acids of the invention are non-naturally occurring; that is, they do not exist in nature.

Thus, in a preferred embodiment, the variant protein has an amino acid sequence that differs from a target sequence by at least 1-5% of the residues. That is, the variant proteins of the invention are less than about 97-99% identical to a target amino acid sequence. Accordingly, a protein is a “candidate variant protein” if the overall homology of the protein sequence to the target sequence is preferably less than about 99%, more preferably less than about 98%, even more preferably less than about 97% and mor preferably less than about 95%. In some embodiments, the homology will be as low as about 75-80%.

Homology in this context means sequence similarity or identity, with identity being preferred. As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc. All references cited in this paragraph are incorporated by reference in their entirety.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in: Altschul et al., J. Mol. Biol. 215, 403-410, (1990); Altschul et al., Nucleic Acids Res. 25:3389-3402 (1997); and Karlin et al., Proc. Natl. Acad. Sci. U.S.A. 90:5873-5787 (1993). A particularly useful BLAST program is the WU-BLAST-2 program which was obtained from Altschul et al., Methods in Enzymology, 266:460-480 (1996); http://blast.wustl/edu/blast/README.html/. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucl. Acids Res., 25:3389-3402. Gapped BLAST uses BLOSUM-62 substitution scores; threshold T parameter set to 9; the two-hit method to trigger ungapped extensions; charges gap lengths of k a cost of 10+k; Xᵤ set to 16, and X_{g} set to 40 for database search stage and to 67 for the output stage of the algorithms. Gapped alignments are triggered by a score corresponding to ˜22 bits.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, “percent (%) nucleic acid sequence identity” with respect to the coding sequence of the polypeptides identified herein is defined as the percentage of nucleotide residues in a candidate sequence that are identical with the nucleotide residues in the coding sequence of the target protein. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer amino acids than the target protein, it is understood that in one embodiment, the percentage of sequence identity will be determined based on the number of identical amino acids in relation to the total number of amino acids. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Thus, the variant proteins of the present invention may be shorter or longer than the target protein. Included within the definition of variant proteins are portions or fragments of the target sequence. Fragments of variant proteins are considered variant a proteins if they share a) at least one antigenic epitope; b) have at least the indicated homology; c) and preferably exhibit the biological activity of the target protein.

In a preferred embodiment, as is more fully outlined below, the candidate variant proteins include further amino acid variations, as compared to a target protein, than those outlined herein. In addition, as outlined herein, any of the variations depicted herein may be combined in any way to form additional novel variant proteins.

In addition, candidate variant proteins can be made that are longer than the target protein, for example, by the addition of other sequences, such as purification tags, fusion sequences, etc, as described in U.S. Ser. No. 09/798,789, incorporated herein by reference in its entirety. For example, the variant proteins of the invention may be fused to other therapeutic proteins or to other proteins such as Fc or serum albumin for pharmacokinetic purposes. See for example U.S. Pat. No. 5,766,883 and 5,876,969, both of which are expressly incorporated by reference.

Also included within the invention are variant proteins comprising variable residues in core, surface, and boundary residues.

In a preferred embodiment, the variant proteins of the invention are human conformers. By “conformer” herein is meant a protein that has a protein backbone 3D structure that is virtually the same but has significant differences in the amino acid side chains. That is, the variant proteins of the invention define a conformer set, wherein all of the proteins of the set share a backbone structure and yet have sequences that differ by at least 1-3-5%. The three dimensional backbone structure of a variant protein thus substantially corresponds to the three dimensional backbone structure of human target protein.

“Backbone” in this context means the non-side chain atoms: the nitrogen, carbonyl carbon and oxygen, and the α-carbon, and the hydrogens attached to the nitrogen and α-carbon. To be considered a conformer, a protein must have backbone atoms that are no more than 2 Å from the human target protein structure, with no more than 1.5 Å being preferred, and no more than 1 Å being particularly preferred. In general, these distances may be determined in two ways. In one embodiment, each potential conformer is crystallized and its three dimensional structure determined. Alternatively, as the former is technically challenging, the sequence of each potential conformer is run in the PDA program to determine whether it is a conformer.

Candidate variant proteins may also be identified as being encoded by candidate variant nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred.

In a preferred embodiment, a candidate variant nucleic acid encodes a candidate variant protein. As will be appreciated by those in the art, due to the degeneracy of the genetic code, an extremely large number of nucleic acids may be made, all of which encode the variant proteins of the present invention. Thus, having identified a particular amino acid sequence, those skilled in the art could make any number of different nucleic acids, by simply modifying the sequence of one or more codons in a way which does not change the amino acid sequence of the variant protein.

In one embodiment, the nucleic acid homology is determined through hybridization studies. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

The candidate variant proteins and nucleic acids of the present invention are recombinant. As used herein, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequence depicted in FIG. 6 also includes the complement of the sequence. By the term “recombinant nucleic acid” herein is meant nucleic acid, originally formed in vitro, in general, by the manipulation of nucleic acid by endonucleases, in a form not normally found in nature. Thus an isolated candidate variant nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a candidate variant protein from one organism in a different organism or host cell. Alternatively, the protein may be made at a significantly higher concentration than is normally seen, through the use of a inducible promoter or high expression promoter, such that the protein is made at increased concentration levels. Furthermore, all of the variant proteins outlined herein are in a form not normally found in nature, as they contain amino acid substitutions, insertions and deletions, with substitutions being preferred, as discussed below.

Also included within the definition of candidate variant proteins of the present invention are amino acid sequence variants of the candidate variant sequences outlined herein. That is, the candidate variant proteins may contain additional variable positions as compared to the target protein. These variants fall into one or more of three classes: substitutional, insertional or deletional variants. These variants ordinarily are prepared by site specific mutagenesis of nucleotides in the DNA encoding a candidate variant protein, using cassette or PCR mutagenesis or other techniques well known in the art, to produce DNA encoding the variant, and thereafter expressing the DNA in recombinant cell culture as outlined above. However, candidate variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the candidate variant protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variant proteins screened for the optimal combination of desired activity. Techniques for making substitution mutations at predetermined sites in DNA having a known sequence are well known, for example, M13 primer mutagenesis and PCR mutagenesis.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the variant protein are desired, substitutions are generally made in accordance with the following chart:

Chart 1

| Original Residue | Exemplary Substitutions |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity, however the immune response may be altered from that of the original candidate variant protein, as needed. Alternatively, the variant may be designed such that the biological activity of the candidate variant protein is altered. For example, glycosylation sites may be altered or removed. Similarly, the biological function may be altered.

In addition, in some embodiments, it is desirable to have candidate variant proteins with altered immunogenicity that are more stable than the target protein. Preferably, it would be desirable have proteins that exhibit oxidative stability, alkaline stability, and thermal stability.

A change in oxidative stability is evidenced by at least about 20%, more preferably at least about 50% increase of activity of a variant protein when exposed to various oxidizing conditions as compared to that of wild-type protein. Oxidative stability is measured by known procedures.

A change in alkaline stability is evidenced by at least about a 5% or greater increase or decrease (preferably increase) in the half life of the activity of a variant protein when exposed to increasing or decreasing pH conditions as compared to that of wild-type protein. Generally, alkaline stability is measured by known procedures.

A change in thermal stability is evidenced by at least about a 5% or greater increase or decrease (preferably increase) in the half life of the activity of a variant protein when exposed to a relatively high temperature and neutral pH as compared to that of wild-type protein. Generally, thermal stability is measured by known procedures.

The candidate variant proteins and nucleic acids of the invention can be made in a number of ways. Individual nucleic acids and proteins can be made as known in the art and outlined below. Alternatively, libraries of candidate variant proteins can be made for testing.

In a preferred embodiment, the library of candidate variant proteins is generated from a probability distribution table. As outlined herein, there are a variety of methods of generating a probability distribution table, including using PDA™ technology, sequence alignments, forcefield calculations such as self-consistent meant field (SCMF) calculations, etc. In addition, the probability distribution can be used to generate information entropy scores for each position, as a measure of the mutational frequency observed in the library.

In this embodiment, the frequency of each amino acid residue at each variable position in the list is identified. Frequencies can be thresholded, wherein any variant frequency lower than a cutoff is set to zero. This cutoff is preferably about 1%, 2%, 5%, 10% or 20%, with about 10% being particularly preferred. These frequencies are then built into the library of candidate variant proteins. That is, as above, these variable positions are collected and all possible combinations are generated, but the amino acid residues that “fill” the library of candidate variant proteins are utilized on a frequency basis. Thus, in a non-frequency based library of candidate variant proteins, a variable position that has 5 possible residues will have about 20% of the proteins comprising that variable position with the first possible residue, 20% with the second, etc. However, in a frequency based library of candidate variant proteins, a variable position that has 5 possible residues with frequencies of about 10%, 15%, 25%, 30% and 20%, respectively, will have 10% of the proteins comprising that variable position with the first possible residue, 15% of the proteins with the second residue, 25% with the third, etc. As will be appreciated by those in the art, the actual frequency may depend on the method used to actually generate the proteins; for example, exact frequencies may be possible when the proteins are synthesized. However, when the frequency-based primer system outlined below is used, the actual frequencies at each position will vary, as outlined below.

As will be appreciated by those in the art and outlined herein, probability distribution tables can be generated in a variety of ways. In addition to the methods outlined herein, self-consistent mean field (SCMF) methods can be used in the direct generation of probability tables. SCMF is a deterministic computational method that uses a mean field description of rotamer interactions to calculate energies. A probability table generated in this way can be used to create libraries of candidate variant proteins as described herein. SCMF can be used in three ways: the frequencies of amino acids and rotamers for each amino acid are listed at each position; the probabilities are determined directly from SCMF (see Delarue et la. Pac. Symp. Biocomput. 109-21 (1997), expressly incorporated by reference). In addition, highly variable positions and non-variable positions can be identified.

Alternatively, another method is used to determine what sequence is jumped to during a search of sequence space; SCMF is used to obtain an accurate energy for that sequence; this energy is then used to rank it and create a rank-ordered list of sequences (similar to a Monte Carlo sequence list). A probability table showing the frequencies of amino acids at each position can then be calculated from this list (Koehl et al., J. Mol. Biol. 239:249 (1994); Koehl et al., Nat. Struc. Biol. 2:163 (1995);

Koehl et al., Curr. Opin. Struct. Biol. 6:222 (1996); Koehl et al., J. Mol. Bio. 293:1183 (1999); Koehl et al., J. Mol. Biol. 293:1161 (1999); Lee J. Mol. Biol. 236:918 (1994); and Vasquez Biopolymers 36:53-70 (1995); all of which are expressly incorporated by reference. Similar methods include, but are not limited to, OPLS-AA (Jorgensen, et al., J. Am. Chem. Soc. (1996), v 118, pp 11225-11236; Jorgensen, W. L.; BOSS, Version 4.1; Yale University: New Haven, CT (1999)); OPLS (Jorgensen, et al., J. Am. Chem. Soc. (1988), v 110, pp 1657ff; Jorgensen, et al., J Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. NatI. Acad. Sci. USA (1999), v 96, pp5482-5485); ECEPP/3 (Liwo et al., J Protein Chem May 1994;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al.,(1988) Proteins: Structure, Function and Genetics, v4,pp31-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.).

In addition, as outlined herein, a preferred method of generating a probability distribution table is through the use of sequence alignment programs. In addition, the probability table can be obtained by a combination of sequence alignments and computational approaches. For example, one can add amino acids found in the alignment of homologous sequences to the result of the computation. Preferable one can add the wild type amino acid identity to the probability table if it is not found in the computation.

As will be appreciated, a library of candidate variant proteins created by recombining variable positions and/or residues at the variable position may not be in a rank-ordered list. In some embodiments, the entire list may just be made and tested. Alternatively, in a preferred embodiment, the secondary library is also in the form of a rank ordered list. This may be done for several reasons, including the size of the secondary library is still too big to generate experimentally, or for predictive purposes. This may be done in several ways. In one embodiment, the secondary library is ranked using the scoring functions of PDA to rank the library members. Alternatively, statistical methods could be used. For example, the secondary library may be ranked by frequency score; that is, proteins containing the most of high frequency residues could be ranked higher, etc. This may be done by adding or multiplying the frequency at each variable position to generate a numerical score. Similarly, the secondary library different positions could be weighted and then the proteins scored; for example, those containing certain residues could be arbitrarily ranked.

In a preferred embodiment, the different protein members of the candidate variant library may be chemically synthesized. This is particularly useful when the designed proteins are short, preferably less than 150 amino acids in length, with less than 100 amino acids being preferred, and less than 50 amino acids being particularly preferred, although as is known in the art, longer proteins can be made chemically or enzymatically. See for example Wilken et al, Curr. Opin. Biotechnol. 9:412-26 (1998), hereby expressly incorporated by reference.

In a preferred embodiment, particularly for longer proteins or proteins for which large samples are desired, the candidate variant sequences are used to create nucleic acids such as DNA which encode the member sequences and which can then be cloned into host cells, expressed and assayed, if desired. Thus, nucleic acids, and particularly DNA, can be made which encodes each member protein sequence. This is done using well known procedures. The choice of codons, suitable expression vectors and suitable host cells will vary depending on a number of factors, and can be easily optimized as needed.

In a preferred embodiment, multiple PCR reactions with pooled oligonucleotides is done, as is generally depicted in FIG. 1. In this embodiment, overlapping oligonucleotides are synthesized which correspond to the full length gene. Again, these oligonucleotides may represent all of the different amino acids at each variant position or subsets.

In a preferred embodiment, these oligonucleotides are pooled in equal proportions and multiple PCR reactions are performed to create full length sequences containing the combinations of mutations defined by the secondary library. In addition, this may be done using error-prone PCR methods.

In a preferred embodiment, the different oligonucleotides are added in relative amounts corresponding to the probability distribution table. The multiple PCR reactions thus result in full length sequences with the desired combinations of mutation in the desired proportions.

The total number of oligonucleotides needed is a function of the number of positions being mutated and the number of mutations being considered at these positions: (number of oligos for constant positions)+M1+M2+M3+ . . . Mn=(total number of oligos required), where Mn is the number of mutations considered at position n in the sequence.

In a preferred embodiment, each overlapping oligonucleotide comprises only one position to be varied; in alternate embodiments, the variant positions are too close together to allow this and multiple variants per oligonucleotide are used to allow complete recombination of all the possibilities. That is, each oligo can contain the codon for a single position being mutated, or for more than one position being mutated. The multiple positions being mutated must be close in sequence to prevent the oligo length from being impractical. For multiple mutating positions on an oligonucleotide, particular combinations of mutations can be included or excluded in the library by including or excluding the oligonucleotide encoding that combination. For example, as discussed herein, there may be correlations between variable regions; that is, when position X is a certain residue, position Y must (or must not) be a particular residue. These sets of variable positions are sometimes referred to herein as a “cluster”. When the clusters are comprised of residues close together, and thus can reside on one oligonuclotide primer, the clusters can be set to the “good” correlations, and eliminate the bad combinations that may decrease the effectiveness of the library. However, if the residues of the cluster are far apart in sequence, and thus will reside on different oligonuclotides for synthesis, it may be desirable to either set the residues to the “good” correlation, or eliminate them as variable residues entirely. In an alternative embodiment, the library may be generated in several steps, so that the cluster mutations only appear together. This procedure, i.e., the procedure of identifying mutation clusters and either placing them on the same oligonucleotides or eliminating them from the library or library generation in several steps preserving clusters, can considerably enrich the experimental library with properly folded protein. Identification of clusters can be carried out by a number of ways, e.g. by using known pattern recognition methods, comparisons of frequencies of occurrence of mutations or by using energy analysis of the sequences to be experimentally generated (for example, if the energy of interaction is high, the positions are correlated). These correlations may be positional correlations (e.g. variable positions 1 and 2 always change together or never change together) or sequence correlations (e.g. if there is a residue A at position 1, there is always residue B at position 2). See: Pattern discovery in Biomolecular Data: Tools, Techniques, and Applications; edited by Jason T. L. Wang, Bruce A. Shapiro, Dennis Shasha. New York: Oxford Unviersity, 1999; Andrews, Harry C. Introduction to mathematical techniques in patter recognition; New York, Wiley-Interscience [1972]; Applications of Pattern Recognition; Editor, K. S. Fu. Boca Raton, Fla. CRC Press, 1982; Genetic Algorithms for Pattern Recognition; edited by Sankar K. Pal, Paul P. Wang. Boca Raton CRC Press, c1996; Pandya, Abhijit S., Pattern recognition with Neural networks in C++/Abhijit S. Pandya, Robert B. Macy. Boca Raton, Fla.: CRC Press, 1996; Handbook of pattern recognition and computer vision/edited by C. H. Chen, L. F. Pau, P. S. P. Wang. 2^{nd} ed. Signapore; River Edge, N.J. World Scientific, c1999; Friedman, Introduction to Pattern Recognition: Statistical, Structural, Neural, and Fuzzy Logic Approaches; River Edge, N.J.: World Scientific, c1999, Series title: Serien a machine perception and artificial intelligence; vol. 32; all of which are expressly incorporated by reference. In addition programs used to search for consensus motifs can be used as well.

In addition, correlations and shuffling can be fixed or optimized by altering the design of the oligonucleotides; that is, by deciding where the oligonucleotides (primers) start and stop (e.g. where the sequences are “cut”). The start and stop sites of oligos can be set to maximize the number of clusters that appear in single oligonucleotides, thereby enriching the library with higher scoring sequences. Different oligonucleotides start and stop site options can be computationally modeled and ranked according to number of clusters that are represented on single oligos, or the percentage of the resulting sequences consistent with the predicted libarary of sequences.

The total number of oligonucleotides required increases when multiple mutable positions are encoded by a single oligonucleotide. The annealed regions are the ones that remain constant, i.e. have the sequence of the reference sequence.

Oligonucleotides with insertions or deletions of codons can be used to create a library expressing different length proteins. In particular computational sequence screening for insertions or deletions can result in secondary libraries defining different length proteins, which can be expressed by a library of pooled oligonucleotide of different lengths.

In a preferred embodiment, the secondary library is done by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are expressly incorporated by reference in their entirety. This set of sequences can also be an artificial set; for example, from a probability table (for example generated using SCMF) or a Monte Carlo set. Similarly, the “family” can be the top 10 and the bottom 10 sequences, the top 100 sequence, etc. This may also be done using error-prone PCR.

Thus, in a preferred embodiment, in silico shuffling is done using the computational methods described therein. That is, starting with either two libraries or two sequences, random recombinations of the sequences can be generated and evaluated.

In a preferred embodiment, error-prone PCR is done to generate the secondary library. See U.S. Pat. Nos. 5,605,793, 5,811,238, and 5,830,721, all of which are hereby incorporated by reference.

This can be done on the optimal sequence or on top members of the library, or some other artificial set or family. In this embodiment, the gene for the optimal sequence found in the computational screen of the primary library can be synthesized. Error prone PCR is then performed on the optimal sequence gene in the presence of oligonucleotides that code for the mutations at the variant positions of the secondary library (bias oligonucleotides). The addition of the oligonucleotides will create a bias favoring the incorporation of the mutations in the secondary library. Alternatively, only oligonucleotides for certain mutations may be used to bias the library.

In a preferred embodiment, gene shuffling with error prone PCR can be performed on the gene for the optimal sequence, in the presence of bias oligonucleotides, to create a DNA sequence library that reflects the proportion of the mutations found in the secondary library. The choice of the bias oligonucleotides can be done in a variety of ways; they can chosen on the basis of their frequency, i.e. oligonucleotides encoding high mutational frequency positions can be used; alternatively, oligonucleotides containing the most variable positions can be used, such that the diversity is increased; if the secondary library is ranked, some number of top scoring positions can be used to generate bias oligonucleotides; random positions may be chosen; a few top scoring and a few low scoring ones may be chosen; etc. What is important is to generate new sequences based on preferred variable positions and sequences.

In a preferred embodiment, PCR using a wild type gene or target gene can be used, as is schematically depicted in FIG. 1. In this embodiment, a starting gene is used; generally, although this is not required, the gene is the wild type gene. In some cases it may be the gene encoding the global optimized sequence, or any other sequence of the list. In this embodiment, oligonucleotides are used that correspond to the variant positions and contain the different amino acids of the secondary library. PCR is done using PCR primers at the termini, as is known in the art. This provides two benefits; the first is that this generally requires fewer oligonucleotides and can result in fewer errors. In addition, it has experimental advantages in that if the wild type gene is used, it need not be synthesized.

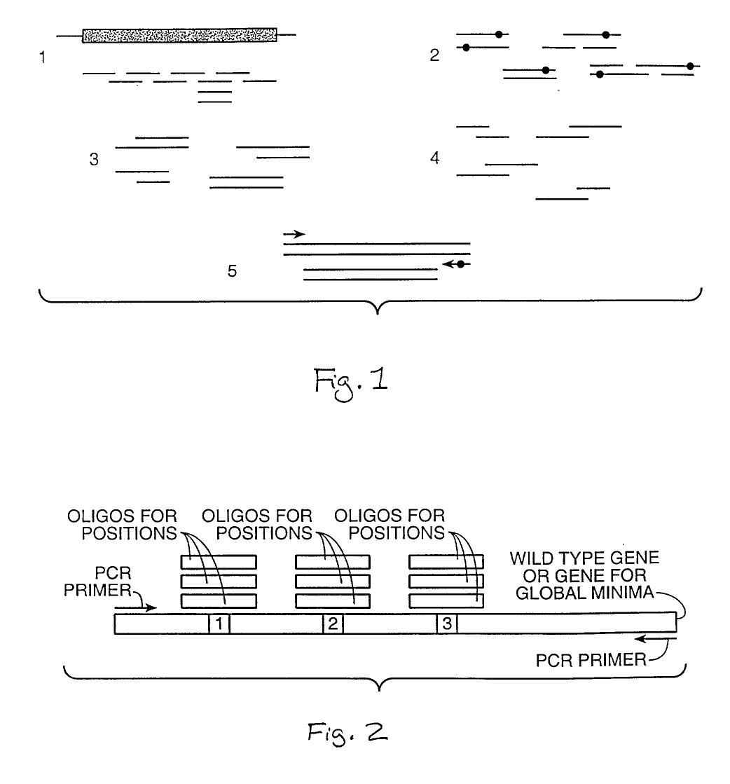

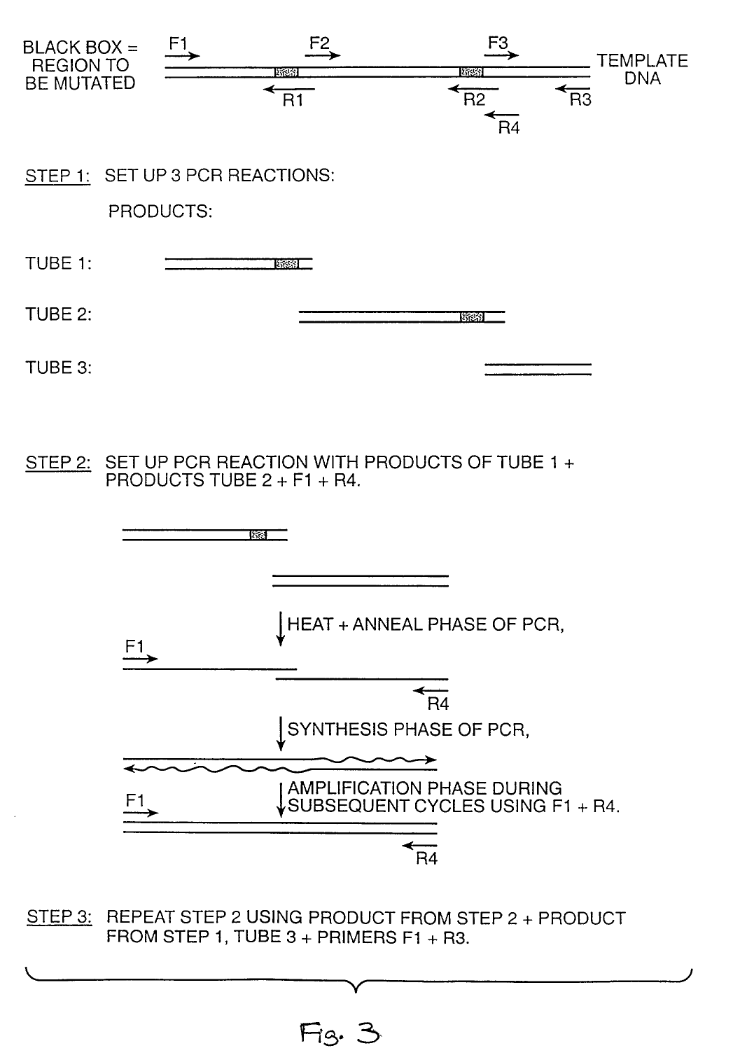

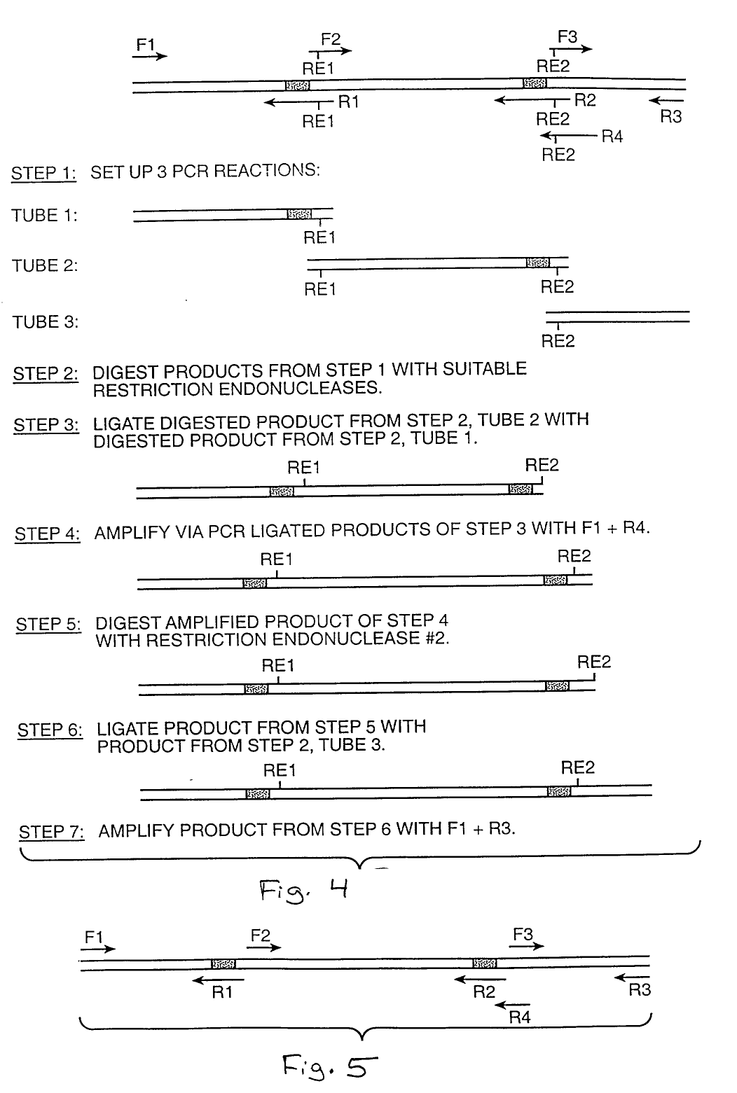

In addition there are several other techniques that can be used as exemplified in FIGS. 2-5. In a preferred embodiment, ligation of PCR products is done.

In a preferred embodiment, a variety of additional steps may be done to one or more candidate variant secondary libraries; for example, further computational processing can occur, candidate variant secondary libraries can be recombined, or cutoffs from different candidate variant secondary libraries can be combined. In a preferred embodiment, a candidate variant secondary library may be computationally remanipulated to form an additional secondary library (sometimes referred to herein as “tertiary libraries”). For example, any of the candidate variant secondary library sequences may be chosen for a second round of PDA, by freezing or fixing some or all of the changed positions in the first secondary library. Alternatively, only changes seen in the last probability distribution table are allowed. Alternatively, the stringency of the probability table may be altered, either by increasing or decreasing the cutoff for inclusion. Similarly, the candidate variant secondary library may be recombined experimentally after the first round; for example, the best gene/genes from the first screen may be taken and gene assembly redone (using techniques outlined below, multiple PCR, error prone PCR, shuffling, etc.). Alternatively, the fragments from one or more good gene(s) to change probabilities at some positions. This biases the search to an area of sequence space found in the first round of computational and experimental screening.

In a preferred embodiment, a tertiary library can be generated from combining candidate variant secondary libraries. For example, a probability distribution table from a candidate variant secondary library can be generated and recombined, wither computationally or experimentally, as outlined herein. A PDA™ technology candidate variant secondary library may be combined with a sequence alignment secondary library, and either recombined (again, computationally or experimentally) or just the cutoffs from each joined to make a new tertiary library. The top sequences from several libraries can be recombined. Primary and secondary libraries can similarly be combined. Sequences from the top of a library can be combined with sequences from the bottom of the library to more broadly sample sequence space, or only sequences distant from the top of the library can be combined. Candidate variant secondary libraries that analyzed different parts of the protein can be combined to a tertiary library that treats the combined parts of the protein.

In a preferred embodiment, a tertiary library can be generated using correlations in the candidate variant secondary library. That is, a residue at a first variable position may be correlated to a residue at second variable position (or correlated to residues at additional positions as well). For example, two variable positions may sterically or electrostatically interact, such that if the first residue is X, the second residue must be Y. This may be either a positive or negative correlation.

Using the nucleic acids of the present invention which encode candidate variant library members, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the library protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the library protein, as will be appreciated by those in the art; for example, transcriptional and translational regulatory nucleic acid sequences from Bacillus are preferably used to express the library protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences include constitutive and inducible promoter sequences. The promoters may be either naturally occurring promoters, hybrid or synthetic promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers resistance to a selection agent. Suitable selection agents include, but are not limited to, neomycin (or its analog G418), blasticidin S, histinidol D, bleomycin, puromycin, hygromycin B, and other drugs.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

A preferred expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al.,Proc. Natl. Acad. Sci. U.S.A., 93:5185-90; Choate et al., Human Gene Therapy, 7:2247 (1996); PCT/US97/01019 and PCT/US97/01048, and references cited therein, all of which are hereby expressly incorporated by reference.

The candidate variant library proteins of the present invention are produced by culturing a host cell transformed with nucleic acid, preferably an expression vector, containing nucleic acid encoding an library protein, under the appropriate conditions to induce or cause expression of the library protein. The conditions appropriate for candidate variant library protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

As will be appreciated by those in the art, the type of cells used in the present invention can vary widely. Basically, a wide variety of appropriate host cells can be used, including yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells. See the ATCC cell line catalog, hereby expressly incorporated by reference. In addition, the expression of the secondary libraries in phage display systems, such as are well known in the art, are particularly preferred, especially when the secondary library comprises random peptides. In one embodiment, the cells may be genetically engineered, that is, contain exogeneous nucleic acid, for example, to contain target molecules.

In a preferred embodiment, the candidate variant library proteins are expressed in mammalian cells. Any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen will be set up such that the cells exhibit a selectable phenotype in the presence of a random library member. As is more fully described below, cell types implicated in a wide variety of disease conditions are particularly useful, so long as a suitable screen may be designed to allow the selection of cells that exhibit an altered phenotype as a consequence of the presence of a library member within the cell.

Accordingly, suitable mammalian cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

Mammalian expression systems are also known in the art, and include retroviral systems. A mammalian promoter is any DNA sequence capable of binding mammalian RNA polymerase and initiating the downstream (3′) transcription of a coding sequence for library protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, candidate variant library proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of library protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the library protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli Streptococcus cremoris, and Streptococcus lividans, among others.

The bacterial expression vectors are transformed into bacterial host cells using techniques well known in the art, such as calcium chloride treatment, electroporation, and others.

In one embodiment, candidate variant library proteins are produced in insect cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In a preferred embodiment, candidate variant library protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

The candidate variant library protein may also be made as a fusion protein, using techniques well known in the art. Thus, for example, for the creation of monoclonal antibodies, if the desired epitope is small, the library protein may be fused to a carrier protein to form an immunogen. Alternatively, the library protein may be made as a fusion protein to increase expression, or for other reasons. For example, when the library protein is an library peptide, the nucleic acid encoding the peptide may be linked to other nucleic acid for expression purposes. Similarly, other fusion partners may be used, such as targeting sequences which allow the localization of the library members into a subcellular or extracellular compartment of the cell, rescue sequences or purification tags which allow the purification or isolation of either the library protein or the nucleic acids encoding them; stability sequences, which confer stability or protection from degradation to the library protein or the nucleic acid encoding it, for example resistance to proteolytic degradation, or combinations of these, as well as linker sequences as needed.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signalling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations or to the outside of the cell via secretion.

In a preferred embodiment, the candidate variant library member comprises a rescue sequence. A rescue sequence is a sequence which may be used to purify or isolate either the candidate agent or the nucleic acid encoding it. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

In a preferred embodiment, the fusion partner is a stability sequence to confer stability to the library member or the nucleic acid encoding it. Thus, for example, peptides may be stabilized by the incorporation of glycines after the initiation methionine (MG or MGGO), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, preferred stability sequences are as follows: MG(X)nGGPP, where X is any amino acid and n is an integer of at least four.

In one embodiment, the candidate variant library nucleic acids, proteins and antibodies of the invention are labeled. By “labeled” herein is meant that nucleic acids, proteins and antibodies of the invention have at least one element, isotope or chemical compound attached to enable the detection of nucleic acids, proteins and antibodies of the invention. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

In a preferred embodiment, the candidate variant library protein is purified or isolated after expression. Library proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the library protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the library protein. In some instances no purification will be necessary.

In a preferred embodiment, the candidate variant protein is purified or isolated after expression. Variant proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the variant protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the variant protein. In some instances no purification will be necessary.

Once expressed and purified if necessary, the candidate variant library proteins and nucleic acids can be tested for altered immunogenicty. Suitable methods include measuring of the binding of MHC peptide complexes to TCRs, measurement of MHC/peptide interactions(Sidney, J., et al., In Current Protocols in Immunology (1998) 18.3.1-18.3.19, the testing of potential T cell epitopes in transgenic mice expressing human MHC molecules, the testing of potential T cell epitopes in mice reconstituted with human antigen-presenting cells and T cell in place of their endogenous cells (WO 98/52976; WO 00/34317), T cell proliferation and CTL assays (Hemmer, B., (1998) J. Immunol., 160:3631-3636), and the “i-mune assay” (Genecor; The Scientist, 15:14, (2001)).

Once made, the candidate variant proteins and nucleic acids of the invention find use in a number of applications. In a preferred embodiment, candidate variant proteins that are less immunogenic than the target protein are used as therapeutic proteins. For example, clinical and preclinical therapy studies have shown that exogenous proteins can be effective in vivo as artificial receptors for the capture of radionuclides, as toxins, or as catalysts for the activation of pro-drugs (Meyer, DL., et al. (2001) Protein Science, 10:491-503). Other uses for therapeutic proteins with reduced immunogenicity includes thrombolytic therapy of acute myocardial infarction (Laroche, Y., et al., (2000) Blood, 96:1425-1432).

In a preferred embodiment, candidate variant proteins that are more immunogenetic than the target protein are used in the development of vaccines and immunotherapeutics for autoimmune disease and cancer. For example, vaccines can be made that are more effective at inducing an immune response by inserting a linear amino acid sequence epitope that has increased affinity for MHC class I or class II molecules (see for example, Sarobe, P., et al. (1998) J. Clin. Invest., 102:1239-1248; Thimme, R., et al. (2001) J. Virology, 75:3984-3987; Roberts, C., et al., (1996) Aids Research and Human Retroviruses, 12:593-610). In other embodiments, vaccines are made that are more effective at inducing an immune response by inserting a sequence encoding a conformational three dimensional epitope that interacts with membrane bound antibodies on naive B cells.

Preferably vaccines are made against Lymes disease, Hepatitis B, Hepatitis C, Poliovirus, and HIV.

In other embodiments, the candidate variant proteins are more immunogenic toward tumor cells.

In a preferred embodiment, a therapeutically effective dose of a candidate variant protein is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 5 μg/kg are used, administered intraveneously, peritoneally, or subcutaneously. As is known in the art, adjustments for candidate variant protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The term “treatment” in the instant invention is meant to include therapeutic treatment, as well as prophylactic, or suppressive measures for the disease or disorder. Thus, for example, successful administration of a candidate variant protein prior to onset of the disease results in “treatment” of the disease. As another example, successful administration of a variant protein after clinical manifestation of the disease to combat the symptoms of the disease comprises “treatment” of the disease. “Treatment” also encompasses administration of a variant protein after the appearance of the disease in order to eradicate the disease. Successful administration of an agent after onset and after clinical symptoms have developed, with possible abatement of clinical symptoms and perhaps amelioration of the disease, comprises “treatment” of the disease.

Those “in need of treatment” include mammals already having the disease or disorder, as well as those prone to having the disease or disorder, including those in which the disease or disorder is to be prevented.

The administration of the candidate variant proteins of the present invention, preferably in the form of a sterile aqueous solution, can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds, inflammation, etc., the candidate variant protein may be directly applied as a solution or spray. Depending upon the manner of introduction, the pharmaceutical composition may be formulated in a variety of ways. The concentration of the therapeutically active candidate variant protein in the formulation may vary from about 0.1 to 100 weight %. In another preferred embodiment, the concentration of the candidate variant protein is in the range of 0.003 to 1.0 molar, with dosages from 0.03, 0.05, 0.1, 0.2, and 0.3 millimoles per kilogram of body weight being preferred.

The pharmaceutical compositions of the present invention comprise a candidate variant protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. See for example, Goodman and Gilman, incorporated herein by reference in its entirety.

In a further embodiment, the candidate variant proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

In one embodiment provided herein, antibodies, including but not limited to monoclonal and polyclonal antibodies, are raised against variant proteins using methods known in the art (see Soren, M., et al (1997) EP 0 752 886; incorporated herein by reference in its entirety). In a preferred embodiment, these anti-variant antibodies are used for immunotherapy. Thus, methods of immunotherapy are provided. By “immunotherapy” is meant treatment of an autoimmune disease associated with the production of self-proteins. In particular, self-proteins are conjugated to a T cell epitope to make an autovaccine. Self proteins of use in the present invention include TNFα, and γ-interferon for the treatment of cancer, IGE for the treatment of allergy, and TNFα, TNFβ, and or interleukin 1 for the treatment of chronic inflammatory diseases.

As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with a variant protein antigen comprising a T cell epitope and a self-protein to which antibodies are raised. As appreciated by one of ordinary skill in the art, the variant protein antigen may be provided by injecting a variant polypeptide against which antibodies are desired to be raised into a recipient, or contacting the recipient with a variant protein encoding nucleic acid, capable of expressing the variant protein antigen, under conditions for expression of the variant TNF-α protein antigen.

In a preferred embodiment, candidate variant proteins are administered as therapeutic agents, and can be formulated as outlined above. Similarly, candidate variant genes (including both the full-length sequence, partial sequences, or regulatory sequences of the variant coding regions) can be administered in gene therapy applications, as is known in the art. These variant genes can include antisense applications, either as gene therapy (i.e. for incorporation into the genome) or as antisense compositions, as will be appreciated by those in the art.

In a preferred embodiment, the nucleic acid encoding the candidate variant proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. [Zamecnik et al., Proc. Natl. Acad. Sci. U.S.A. 83:4143-4146 (1986)]. The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection [Dzau et al., Trends in Biotechnology 11:205-210 (1993)]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

In a preferred embodiment, candidate variant genes are administered as DNA vaccines, either single genes or combinations of candidate variant genes. Naked DNA vaccines are generally known in the art. Brower, Nature Biotechnology, 16:1304-1305 (1998). Methods for the use of genes as DNA vaccines are well known to one of ordinary skill in the art, and include placing a candidate variant gene or portion of a variant gene under the control of a promoter for expression in a patient in need of treatment. The variant gene used for DNA vaccines can encode full-length variant proteins, but more preferably encodes portions of the variant proteins including peptides derived from the variant protein. In a preferred embodiment a patient is immunized with a DNA vaccine comprising a plurality of nucleotide sequences derived from a variant gene. Similarly, it is possible to immunize a patient with a plurality of variant genes or portions thereof as defined herein. Without being bound by theory, expression of the polypeptide encoded by the DNA vaccine, cytotoxic T-cells, helper T-cells and antibodies are induced which recognize and destroy or eliminate cells expressing TNF-α proteins.

In a preferred embodiment, the DNA vaccines include a gene encoding an adjuvant molecule with the DNA vaccine. Such adjuvant molecules include cytokines that increase the immunogenic response to the variant polypeptide encoded by the DNA vaccine. Additional or alternative adjuvants are known to those of ordinary skill in the art and find use in the invention.

All references cited herein are incorporated by reference.

We claim:

1. A method for modulating the immunogenicity of a target protein, said method comprising:
a) inputting a protein backbone structure with variable residue positions of a target protein into a computer;
b) computationally generating a set of primary variant amino acid sequences; and,
c) applying a computational immunogenicity filter against said set to identify at least one candidate variant protein.

2. A method according to claim 1 further comprising testing said candidate variant protein to determine if said immunogenicity is altered relative to said target protein.

3. A method according to claim 1 further comprising classifying each variable residue position as either a core, surface or boundary residue.

4. A method according to claim 1 wherein said computationally generating step comprises a DEE computation.

5. A method according to claim 4 wherein said DEE computation is selected from the group consisting of original DEE and Goldstein DEE.

6. A method according to claim 1 wherein said set of primary variant amino acid sequences are optimized for at least one scoring function.

7. A method according to claim 6 wherein said set of primary variant amino sequences optimized for at least one scoring function comprises the globally optimal protein sequence.

8. A method according to claim 6 wherein said scoring function is selected from the group consisting of a Van der Waals potential scoring function, a hydrogen bond potential scoring function, an atomic salvation scoring function, an electrostatic scoring function and a secondary structure propensity scoring function.

9. A method according to claim 1 wherein said computationally generating step includes the use of a Monte Carlo search.

10. A method according to claim 1 wherein said target protein is from a non human species and said candidate variant protein exhibits reduced immunogenicity in humans.

11. A method according to claim 1 wherein the immunogenicity of said candidate variant protein is reduced relative to said target protein.

12. A method according to claim 1 wherein said candidate variant protein is non-immunogenic.

13. A method according to claim 11 or 12 wherein said candidate variant protein is more stable than said target protein.

14. A method according to claim 1 wherein said modulating the immunogenicity of said target protein comprises modifying the amino acid sequence that binds to an MHC molecule.

15. A method according to claim 14 wherein said MHC molecule belongs to MHC class I.

16. A method according to claim 14 wherein said MHC molecule belongs to MHC class II.

17. A method according to claim 1 wherein said modulating the immunogenicity of said target protein comprises modifying an amino acid sequence encoding a T cell epitope.

18. A method for modulating the immunogenicity of a target protein, said method comprising:
a) inputting a protein backbone structure with variable residue positions of a target protein into a computer;
b) applying a computational immunogenicity filter to identify at least one candidate variant protein;
d) computationally analyzing said variant protein for maintenance of native fold and stability; nd
d) generating a set of primary variant amino acid sequences.
